SEQUENCE LISTING (42 sequences)

SEQ ID NO: 1 - RNA, 66 nt, Mus musculus
agggcuccugacuccagguccuguguguuaccucgaaauagcacuggacuuggagucagaaggccu

SEQ ID NO: 2 - RNA, 65 nt, Mus musculus
cauaaacccguagauccgaucuuguggugaaguggaccgcgcaagcucguuucuaugggucugug

SEQ ID NO: 3 - RNA, 77 nt, Mus musculus
ugcgcuccccucagucccugagacccuaacuugugauguuuaccguuuaaauccacggguuaggcucuugggagcug

SEQ ID NO: 4 - RNA, 110 nt, Mus musculus
uggaagcuucaggagauccugcuccgucgccccaguguucagacuaccuguucaggacaaugccguuguacaguagucugcacauugguuagacugggcaagggccagca

SEQ ID NO: 5 - RNA, 110 nt, Mus musculus; identical to SEQ ID NO: 4

SEQ ID NO: 6 - RNA, 128 nt, Mus musculus
cagccagcucugaucucgcccucccugagggguccuguacugagcugccccgagguccuucacugugcucagcucggggcagcucaguacaggaugcgucagggugggagacaacggggaacaagcca

SEQ ID NO: 7 - RNA, 84 nt, Mus musculus
ccugcccccgccccagcagcacacugugguuuguacggcacuguggccacguccaaaccacacugugguguuagagcgagggua

SEQ ID NO: 8 - RNA, 97 nt, Mus musculus
cugucucggagccuggggcaggggggcaggaggggcucagggagaaaguaucuacagccccuggcccucucugcccuuccguccccuguccccaagu

SEQ ID NO: 9 - RNA, 83 nt, Mus musculus
uuccuuugacgggugagcuuuuggcccggguuauaccugacacucacguauaagacgagcaaaaagcuuguuggucagaggag

SEQ ID NO: 10 - RNA, 110 nt, Mus musculus
ccggggcagucccuccaggcucaggacagccacugcccaccgcacacugcguugcuccggacccacugugcgugugacagcggcugaucugucccugggcagcgcgaacc

SEQ ID NO: 11 - RNA, 65 nt, Mus musculus
agggauuggagagaaaggcaguuccugaugguccccucccaggggcuggcuuuccucugguccuu

SEQ ID NO: 12 - RNA, 82 nt, Mus musculus
aagucugugucuguaaacauccccgacuggaagcuguaagccacagccaagcuuucagucagauguuugcugcuacuggcuc

SEQ ID NO: 13 - RNA, 68 nt, Mus musculus
cuccggugccuacugagcugauaucaguucucauuucacacacuggcucaguucagcaggaacaggag

SEQ ID NO: 14 - RNA, 89 nt, Mus musculus
accauguuguaguguguguaaacauccuacacucucagcugugagcucaagguggcugggagaggguuguuuacuccuucugccaugga

SEQ ID NO: 15 - RNA, 79 nt, Mus musculus
gggugggcagcuguuaagacuugcagugauguuuagcuccucugcaugugaacaucacagcaagucugugcugcugccu

SEQ ID NO: 16 - RNA, 88 nt, Mus musculus
aucucuuacacaggcugaccgauuucuccugguguucagagucuguuuuugucuagcaccauuugaaaucgguuaugauguaggggga

SEQ ID NO: 17 - RNA, 64 nt, Mus musculus
gagcucuuuucacauugugcuacugucuaacguguaccgagcagugcaauguuaaaagggcauc

SEQ ID NO: 18 - RNA, 83 nt, Mus musculus
cucaucuugcgguacucaaacuaugggggcacuuuuuuuuuucuuuaaaaagugccgccuaguuuuaagccccgccgguugag

SEQ ID NO: 19 - RNA, 73 nt, Mus musculus
aggugcagagcuuagcugauuggugaacagugauugguuuccgcuuuguucacaguggcuaaguucugcaccu

SEQ ID NO: 20 - RNA, 110 nt, Mus musculus; identical to SEQ ID NO: 4

SEQ ID NO: 21 - RNA, 88 nt, Mus musculus
ugucccguuucucugcuucuacucagaagugcucugagcauagaacuguccuguuugagcagcacuggggaggcagaggcaggaggau

SEQ ID NO: 22 - RNA, 73 nt, Mus musculus
ccgggccuagguucugugauacacuccgacucgggcucuggagcagucagugcaugacagaacuugggcccgg

SEQ ID NO: 23 - RNA, 58 nt, Homo sapiens
cccuguaucccucugaaacacuggaagagcuaguaaauuguaaaugaaguaauacugu

SEQ ID NO: 24 - RNA, 58 nt, Mus musculus
accccuguaucucugaaacacuggaagugcuaguaaauuguaaaugaaauaauacccu

SEQ ID NO: 25 - RNA, 58 nt, Rattus norvegicus
accccuguaucucuaaaacacuggaagagcuaguaaauuguacaugaaauaauacugu

SEQ ID NO: 26 - RNA, 23 nt, Mus musculus
cccaguguucagacuaccuguuc

SEQ ID NO: 27 - RNA, 58 nt, Homo sapiens; identical to SEQ ID NO: 23

SEQ ID NO: 28 - RNA, 58 nt, Mus musculus; identical to SEQ ID NO: 24

SEQ ID NO: 29 - RNA, 58 nt, Rattus norvegicus; identical to SEQ ID NO: 25

SEQ ID NO: 30 - RNA, 23 nt, Mus musculus
cccaguguuuagacuaucuguuc

SEQ ID NO: 31 - RNA, 43 nt, Homo sapiens
cccuacuguguugguggugcugaugaaucugccagaguugagu

SEQ ID NO: 32 - RNA, 41 nt, Mus musculus
cucuauugugcugguggugcugaugaaucugccaauugaau

SEQ ID NO: 33 - RNA, 41 nt, Rattus norvegicus; identical to SEQ ID NO: 32

SEQ ID NO: 34 - RNA, 20 nt, Mus musculus
uagcaccauuugaaaucggu

SEQ ID NO: 35 - RNA, 34 nt, Homo sapiens
ccuggucugcagggccagaggccaggagggugcc

SEQ ID NO: 36 - RNA, 32 nt, Mus musculus
gcugagcuccagggcaaaggcagaagggugcc

SEQ ID NO: 37 - RNA, 33 nt, Rattus norvegicus
gcugaccuccagggcaggggccagaagaguacc

SEQ ID NO: 38 - RNA, 22 nt, Mus musculus
cuggcccucucugcccuuccgu

SEQ ID NO: 39 - RNA, 35 nt, Homo sapiens
ggguuagacagccucagggcaccauuuuuaauccc

SEQ ID NO: 40 - RNA, 33 nt, Mus musculus
gguucugguggccucuggguccguuucugaucc

SEQ ID NO: 41 - RNA, 33 nt, Rattus norvegicus
gguucuggaggccucaggguacguuucugaucc

SEQ ID NO: 42 - RNA, 22 nt, Mus musculus
ucccugagacccuaacuuguga

(19) United States
(12) Patent Application Publication
(10) Pub. No.: US 2011/0160285 A1
(43) Pub. Date: Jun. 30, 2011

(54) IDENTIFICATION OF MIRNA PROFILES THAT ARE DIAGNOSTIC OF HYPERTROPHIC CARDIOMYOPATHY

(75) Inventors: Emily Anderson, Lafayette, CO (US); Anastasia Khvorova, Northborough, MA (US); Anita G. Seto, Golden, CO (US); Scott Baskerville, Louisville, CO (US); Leslie Leinwand, Boulder, CO (US); Kevin G. Sullivan, Boulder, CO (US)

(73) Assignees: THE REGENTS OF THE UNIVERSITY OF COLORADO, Denver, CO (US); Dharmacon, Inc., Lafayette, CO (US)

(21) Appl. No.: 12/921,827

(22) PCT Filed: Mar. 12, 2009
(86) PCT No.: PCT/US09/36939

§ 371 Date: Mar. 10, 2011

Related U.S. Application Data
(60) Provisional application No. 61/069,513, filed on Mar. 13, 2008.

(51) Int. Cl. A61K 31/7105; C40B 30/04; A61P 9/00

(52) U.S. Cl. 514/44 A; 506/9

(57) ABSTRACT

Disclosed herein are a collection of miRNAs and genes whose expression is altered in hypertrophic cardiomyopathy. Accordingly, these miRNAs and genes, singly or in combination, are useful as molecular markers for diagnosis or prognosis of hypertrophic cardiomyopathy. The miRNAs and genes disclosed can also be therapeutic targets for cardiac hypertrophy. For example, agents such as miRNA mimics, miRNA inhibitors or siRNAs for a given miRNA or gene can be used to modulate the level of these molecules thereby inhibiting or preventing hypertrophic cardiomyopathy.

### RELATED APPLICATION INFORMATION

This application is being filed on 12 Mar. 2009, as a PCT International Patent application in the name of Dharmacon, Inc., a U.S. national corporation, applicant for the designation of all countries except the U.S., and Anita G. Seto, a citizen of the U.S., Scott Baskerville, a citizen of the U.S., Leslie Leinwand, a citizen of the U.S., Kevin G. Sullivan, a citizen of the U.S., Emily Anderson, a citizen of the U.S., and Anastasia Khvorova, a citizen of Russia, applicants for the designation of the U.S. only, and claims priority to U.S. Provisional Patent Application Ser. No. 61/069,513 filed on 13 Mar. 2008.

### FIELD OF THE INVENTION

This application relates to the field of treating and diagnosing heart disease, particularly hypertrophic cardiomyopathy.

### BACKGROUND

Hypertrophic cardiomyopathy is the second most common disease of the heart muscle (the myocardium) and is associated with a thickening of the walls of heart. Causes of the disease are diverse. In some cases, symptoms can arise from one or more vascular obstructions. In still other cases, origins are non-obstructive and symptoms are associated with genetic disorders.

Familial hypertrophic cardiomyopathy (hereinafter “FHC”) is an autosomal dominant form of the hypertrophic cardiomyopathy that is observed in approximately 0.2% of the population. At the cellular level, FHC patients exhibit myocyte hypertrophy (enlargement) and a disarray of myofibrils, the bundles of filaments that are responsible for contraction in muscle cells. These abnormalities lead to a wide array of clinical symptoms including dyspnea (difficulty in breathing) and eventual heart failure.

Mutations in a number of genes including myosin heavy chain, troponin-T, and the myosin binding protein C have all been shown to be associated with FHC. The polygenic origins of the disorder suggest that a general defect in the muscle sarcomere (the actin-myosin contractile unit) may be the underlying cause behind this disease.

While there is currently no therapy available for FHC patients, identification of potential therapeutic targets as well as molecular markers that are indicative of hypertrophic cardiomyopathy are imperative for future diagnosis and drug development. The following disclosure identifies multiple markers and drug targets for hypertrophic cardiomyopathy.

### SUMMARY OF THE DISCLOSURE

The present disclosure provides a collection of 22 miRNAs (see Table 1) associated with hypertrophic cardiomyopathy, along with uses thereof.

In one aspect, the disclosure provides a collection of microRNAs (miRNAs) that can be used individually or in combination, or in combination with other indicators, as molecular markers to assess the state of fitness of the heart. Specifically, the miRNA markers disclosed herein can be used as diagnostic and prognostic markers of FHC and other forms of hypertrophic cardiomyopathy.

In another aspect, the disclosure provides miRNAs that can be used individually or in combination, or in combination with other indicators, as prognostic indicators of the effectiveness of a particular treatment for FHC and/or other forms of hypertrophic cardiomyopathy. Alternatively, one or more members of said collection can be used individually or in combination, or in combination with other indicators as molecular markers in screens designed to identify novel drugs for the treatment of FHC and other diseases of hypertrophic cardiomyopathy.

In another aspect, the disclosure provides methods of treating patients with FHC or other forms of hypertrophic cardiomyopathy by modulating the levels of one or more of the miRNAs listed in Table 1 and thereby improving the condition of the patient. In one embodiment, the method comprises modulating the levels of one or more of the miRNAs listed in Table 1 by introducing into patients one or more of the miRNAs, miRNA mimics, or and/or miRNA inhibitors of the miRNAs disclosed herein.

In another aspect, the disclosure provides a collection of genes (see Tables 2-3) associated with FHC and/or other forms of hypertrophic cardiomyopathy, and uses thereof. The genes are either directly-modulated by the miRNAs of Table 1, or are indirectly-modulated by the miRNAs of Table 1.

In another aspect, the disclosure provides a collection of genes (see Tables 2-3) that can be used individually or in combination, or in combination with other indicators as molecular markers to assess the state of fitness of the heart. Specifically, the genes disclosed herein can be used as diagnostic and prognostic markers of FHC and other forms of hypertrophic cardiomyopathy.

In another aspect, the disclosure provides a collection of genes (see Tables 2-3) that can be used individually or in combination, or in combination with other indicators as prognostic indicators of the effectiveness of a particular treatment for FHC and/or other forms of hypertrophic cardiomyopathy. Alternatively, one or more members of said collection can be used individually or in combination, or in combination with other indicators as molecular markers in screens designed to identify novel drugs for the treatment of FHC and other forms of hypertrophic cardiomyopathy.

In another aspect, the disclosure provides methods of treating FHC and/or other forms of hypertrophic cardiomyopathy by modulating the expression of one or more of the genes listed in Tables 2-3 and thereby improving the condition of the patient. For example, one or more of the genes of the disclosure can be over-expressed or down-regulated.

In another aspect, the disclosure provides a method of diagnosing hypertrophic cardiomyopathy comprising, a) measuring the level of expression of a miRNA from Table 1, or an ortholog thereof, in a heart sample from a subject, and b) comparing the level of expression of the miRNA with that of normal heart tissue. If the level of expression of said miRNA in the subject sample is different to the level of expression of the miRNA in normal heart tissue, the subject is determined to have hypertrophic cardiomyopathy. In one embodiment, the miRNA is selected from the group consisting of the human ortholog of mmu-miR-709, the human ortholog of mmu-miR-290, hsa-miR-208a, hsa-miR-185, hsa-miR-30d, hsa-miR-30c, hsa-miR-499, and hsa-miR-29c; if the level of expression of the miRNA in the subject sample is lower than the level of expression of the miRNA in normal heart tissue, then the subject is diagnosed as having hypertrophic cardiomyopathy. In another embodiment, the miRNA is selected from the group consisting of hsa-miR-378, hsa-miR-99a, hsa-miR-125b, hsa-miR-199a-3p, hsa-miR-199b, hsa-miR-486, hsa-miR-497, hsa-miR-328, hsa-miR-210, hsa-miR-24, hsa-miR-130a, hsa-miR-27b, hsa-miR-199a-5p, and hsa-miR-152; if the level of expression of the miRNA in the subject sample is higher than the level of expression of the miRNA in normal heart tissue, then the subject is diagnosed as having hypertrophic cardiomyopathy.

In another aspect, the disclosure provides a method of diagnosing hypertrophic cardiomyopathy comprising, a) measuring the level of expression of a gene from Tables 2-3, or an ortholog thereof, in a heart sample from a subject and b) comparing the level of expression of the gene with that of normal heart tissue, wherein if the level of expression of the gene in the subject sample is different to the level of expression of the gene in normal heart tissue, the subject is determined to have hypertrophic cardiomyopathy. In one embodiment, the gene is selected from the group consisting of ACAA2, ACTR10, ALDOB, BCAR3, C1GALT1, CDH22, DCI, EGF, FBXO31, GFAP, GPR155, GRIN2C, HECTD1, LAMB3, MFSD4, MTRF1L, POLR3A, SAPS3, SLC26A6, TBC1D10C, TFPI, TMEM116, TMEM37, TSPAN6, UNG, and WDR33; if the level of expression of the gene in the subject sample is lower than the level of expression of the gene in normal heart tissue, then the subject is diagnosed as having hypertrophic cardiomyopathy. In another embodiment, the gene is selected from the group consisting of ACTA2, APITD1, CCDC68, CCND2, CFH, COL4A4, COX19, DAPK2, DISP2, EAF2, EFNA5, ENAH, FETUB, GNA15, GNG13, IGFBP6, INMT, LRTOMT, MCM2, MLLT11, MON1B, NLRC3, OMD, PPM1E, PRKAG3, PROCR, RAD51L3, and WISP2; if the level of expression of the gene in the subject sample is higher than the level of expression of the gene in normal heart tissue, then the subject is diagnosed as having hypertrophic cardiomyopathy.

In another aspect, the disclosure provides a method of treating hypertrophic cardiomyopathy comprising a) identifying a subject suspected of having hypertrophic cardiomyopathy, and b) inhibiting the expression or activity of a miRNA selected from the group consisting of hsa-miR-378, hsa-miR-99a, hsa-miR-125b, hsa-miR-199a-3p, hsa-miR-199b, hsa-miR-486, hsa-miR-497, hsa-miR-328, hsa-miR-210, hsa-miR-24, hsa-miR-130a, hsa-miR-27b, hsa-miR-199a-5p, and hsa-miR-152 in the heart cells of the subject. In one embodiment, a miRNA inhibitor is used to inhibit the activity of the miRNA.

In another aspect, the disclosure provides a method of treating hypertrophic cardiomyopathy comprising a) identifying a subject suspected of having hypertrophic cardiomyopathy, and b) increasing the level of a miRNA selected from the group consisting of the human ortholog of mmu-miR-709, the human ortholog of mmu-miR-290, hsa-miR-208a, hsa-miR-185, hsa-miR-30d, hsa-miR-30c, hsa-miR-499, and hsa-miR-29c in the heart cells of the subject. In one embodiment, a miRNA mimic is used to increase the level of the miRNA.

In another aspect, the disclosure provides a method of treating hypertrophic cardiomyopathy comprising a) identifying a subject suspected of having hypertrophic cardiomyopathy, and b) inhibiting the expression or activity of a gene selected from the group consisting of ACTA2, APITD1, CCDC68, CCND2, CFH, COL4A4, COX19, DAPK2, DISP2, EAF2, EFNA5, ENAH, FETUB, GNA15, GNG13, IGFBP6, INMT, LRTOMT, MCM2, MLLT11, MON1B, NLRC3, OMD, PPM1E, PRKAG3, PROCR, RAD51L3, and WISP2 in heart cells of the subject.

In another aspect, the disclosure provides a method of treating hypertrophic cardiomyopathy comprising a) identifying a subject suspected of having hypertrophic cardiomyopathy, and b) increasing the expression or activity of a gene selected from the group consisting of ACAA2, ACTR10, ALDOB, BCAR3, C1GALT1, CDH22, DCI, EGF, FBXO31, GFAP, GPR155, GRIN2C, HECTD1, LAMB3, MFSD4, MTRF1L, POLR3A, SAPS3, SLC26A6, TBC1D10C, TFPI, TMEM116, TMEM37, TSPAN6, UNG, and WDR33 in heart cells of the subject.

These and other aspects and embodiments of the disclosure are described in more detail in the following.

### BRIEF DESCRIPTION OF THE FIGURES

FIG. 1 provides a bar graph depicting the relative levels of twenty-two microRNAs identified in the experiments described in Example 1. Microarray experiments identified miRNAs that were either over-expressed (first fourteen) or under-expressed (last eight) in mutant heart tissues compare to expression in wild type (“WT”) heart tissue. Relative fluorescence intensity values were generated for each microRNA on the microarray followed by log-transformation. Data were averaged across all biological and technical replicates for each genotype and a p-value cut-off value of 0.05 was applied to distinguish differences that were significant. The log difference was calculated by subtracting the log-transformed relative intensity value of the mutant from the wild type value. Plotted is the calculated log difference for each of the twenty-two miRNAs in Table 1.

FIG. 2A-2B provide an example of miR-199a, miR-199b, miR-29c, and miR-328 alignment with 3′ untranslated region (hereinafter “UTR”) target sites identified bioinformatically. The target genes are depicted in 5′ to 3′ orientation, whereas the miRNAs are depicted in 3′ to 5′ orientation.

### DETAILED DESCRIPTION OF THE EMBODIMENTS

All references cited in this disclosure are incorporated into the disclosure in their entirety.

The term “microRNA”, “miRNA”, and “miR” are synonymous and refer to a collection of non-coding RNA molecules which regulate gene expression. miRNAs are found in a wide range of organisms (viruses→humans) and have been shown to play a role in development, homeostasis, and disease etiology. MicroRNAs are processed from single stranded primary transcripts known as pri-miRNA to short stem-loop structures (hairpins) called pre-miRNA and finally to mature miRNA. One or both strands of the mature miRNA molecules are partially complementary to one or more messenger RNA (mRNA) molecules, and function to downregulate gene expression by either cleavage or translation attenuation mechanisms.

The term “mature strand” refers to the strand of a fully processed miRNA, or an siRNA that enters RISC. In some cases, miRNAs have a single mature strand that can vary in length between about 16-31 nucleotides in length. In other instances, miRNAs can have two mature strands (i.e. two unique strands that can enter RISC), and the length of the strands can vary between about 16 and 31 nucleotides. In the present disclosure, the terms “mature strand” and “antisense strand” are used interchangeably.

The terms “microRNA inhibitor”, “miR inhibitor”, or “inhibitor” are synonymous and refer to oligonucleotides or modified oligonucleotides that interfere with the ability of specific miRNAs, or siRNAs to silence their intended targets. Inhibitors can adopt a variety of configurations including single stranded, double stranded, and hairpin designs (see WO2007/095387 for double stranded inhibitor designs). miRNA inhibitors can also include modified nucleotides including but not limited to 2′-O-methyl modified and Locked Nucleic Acid (LNA) modified molecules. See Krutzfeldt et al. 2005. Nature. 438(7068):685-9. In some instances, inhibitors are short (21-31 nucleotides) single stranded, and heavily 2′-O-alkyl modified molecules.

The term “microRNA mimic” refers to synthetic non-coding RNAs that are capable of entering the RNAi pathway and regulating gene expression. miRNA mimics imitate the function of endogenous microRNAs (miRNAs) and can be designed as mature, double stranded molecules or mimic precursors (e.g., pri- or pre-miRNAs). miRNA mimics can be comprised of modified or unmodified RNA, DNA, RNA-DNA hybrids, or alternative nucleic acid chemistries (e.g., LNAs or 2′-O,4′-C-ethylene-bridged nucleic acids (ENA)). For mature, double stranded miRNA mimics, the length of the duplex region can vary between 16 and 31 nucleotides and chemical modification patterns can comprise one or more of the following: the sense strand contains 2′-O-methyl modifications of nucleotides 1 and 2 (counting from the 5′ end of the sense oligonucleotide), and all of the Cs and Us. The antisense strand modifications comprise 2′ F modification of all of the Cs and Us, phosphorylation of the 5′ end of the oligonucleotide, and stabilized internucleotide linkages associated with a 2 nucleotide 3′ overhang. Mimics can also comprise linker conjugate modifications that enhance stability, delivery, specificity, functionality, or strand usage. Preferred microRNA mimics of the disclosure are duplexes formed between a sense strand and an antisense strand where the antisense strand has significant levels of complementarity to both the sense strand and to a target gene, and where:
a. the sense strand ranges in size from about 16 to about 31 nucleotides and nucleotides 1 and 2 (counting from the 5′ end) and all C nucleotides and all U nucleotides in the sense strand are 2′O-methyl modified;
b. the antisense strand ranges in size from about 16 to about 31 nucleotides and all C nucleotides and all U nucleotides in the antisense strand are 2′ F modified;
c. a cholesterol molecule is attached to the 3′ end of the sense strand via a C5 linker molecule such that the sense stand has the following structure (where “oligo” represents the nucleotides of the sense strand):

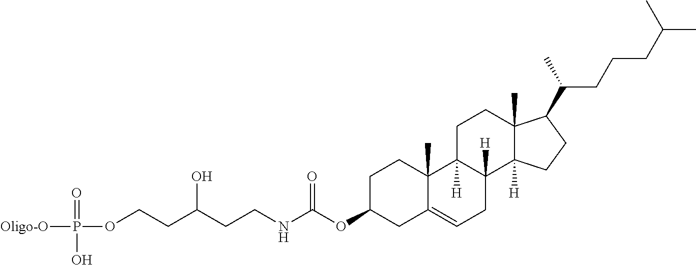

d. a phosphate group is present at the 5′ end of the antisense strand;
e. a 2 nucleotide overhang is present at the 3′ end of the antisense strand comprising phosphorothioate linkages; and
f. a mismatch is present between nucleotide 1 on the antisense strand and the opposite nucleotide on the sense strand and/or a mismatch is present between nucleotide 7 on the antisense strand and the opposite nucleotide on the sense strand and/or a mismatch is present between nucleotide 14 on the antisense strand and the opposite nucleotide on the sense strand (where the specified nucleotide positions are counted from the 5′ end of the antisense strand).

The term “miRNA seed” or “seed” refers to a region of the antisense strand(s) of a microRNA or microRNA mimic. The region generally includes nucleotides 2-6 or 2-7 counting from the 5′ end of the antisense strand.

The term “miRNA seed complement” or “seed complement” refers to a sequence of nucleotides in a target gene, often in the 3′ UTR of a target gene, that is complementary to some or all of the miRNA seed.

The term “gene silencing” refers to a process by which the expression of a specific gene product is lessened or attenuated by RNA interference. The level of gene silencing (also sometimes referred to as the degree of “knockdown”) can be measured by a variety of means, including, but not limited to, measurement of transcript levels by Northern Blot Analysis, B-DNA techniques, transcription-sensitive reporter constructs, expression profiling (e.g. DNA chips), qRT-PCR and related technologies. Alternatively, the level of silencing can be measured by assessing the level of the protein encoded by a specific gene. This can be accomplished by performing a number of studies including Western Analysis, measuring the levels of expression of a reporter protein that has e.g. fluorescent properties (e.g., GFP) or enzymatic activity (e.g. alkaline phosphatases), or several other procedures.

The term “nucleotide” refers to a ribonucleotide or a deoxyribonucleotide or modified form thereof, as well as an analog thereof. Nucleotides include species that comprise purines, e.g., adenine, hypoxanthine, guanine, and their derivatives and analogs, as well as pyrimidines, e.g., cytosine, uracil, thymine, and their derivatives and analogs. Nucleotide analogs include nucleotides having modifications in the chemical structure of the base, sugar and/or phosphate, including, but not limited to, 5-position pyrimidine modifications, 8-position purine modifications, modifications at cytosine exocyclic amines, and substitution of 5-bromo-uracil; and 2′-position sugar modifications, including but not limited to, sugar-modified ribonucleotides in which the 2′-OH is replaced by a group such as an H, OR, R, halo, SH, SR, NH₂, NHR, NR₂, or CN, wherein R is an alkyl moiety. Nucleotide analogs are also meant to include nucleotides with bases such as inosine, queuosine, xanthine, sugars such as 2′-methyl ribose, non-natural phosphodiester linkages such as methylphosphonates, phosphorothioates and peptides.

Modified bases refer to nucleotide bases such as, for example, adenine, guanine, cytosine, thymine, uracil, xanthine, inosine, and queuosine that have been modified by the replacement or addition of one or more atoms or groups. Some examples of types of modifications that can comprise nucleotides that are modified with respect to the base moieties include but are not limited to, alkylated, halogenated, thiolated, aminated, amidated, or acetylated bases, individually or in combination. More specific examples include, for example, 5-propynyluridine, 5-propynylcytidine, 6-methyladenine, 6-methylguanine, N,N-dimethyladenine, 2-propyladenine, 2-propylguanine, 2-aminoadenine, 1-methylinosine, 3-methyluridine, 5-methylcytidine, 5-methyluridine and other nucleotides having a modification at the 5 position, 5-(2-amino)propyl uridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2,2-dimethylguanosine, 5-methylaminoethyluridine, 5-methyloxyuridine, deazanucleotides such as 7-deaza-adenosine, 6-azouridine, 6-azocytidine, 6-azothymidine, 5-methyl-2-thiouridine, other thio bases such as 2-thiouridine and 4-thiouridine and 2-thiocytidine, dihydrouridine, pseudouridine, queuosine, archaeosine, naphthyl and substituted naphthyl groups, any O- and N-alkylated purines and pyrimidines such as N6-methyladenosine, 5-methylcarbonylmethyluridine, uridine 5-oxyacetic acid, pyridine-4-one, pyridine-2-one, phenyl and modified phenyl groups such as aminophenol or 2,4,6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoai nleotides, and alkylcarbonylalkylated nucleotides. Modified nucleotides also include those nucleotides that are modified with respect to the sugar moiety, as well as nucleotides having sugars or analogs thereof that are not ribosyl. For example, the sugar moieties may be, or be based on, mannoses, arabinoses, glucopyranoses, galactopyranoses, 4′-thioribose, and other sugars, heterocycles, or carbocycles.

The term “nucleotide” is also meant to include what are known in the art as universal bases. By way of example, universal bases include, but are not limited to, 3-nitropyrrole, 5-nitroindole, or nebularine. The term “nucleotide” is also meant to include the N3′ to P5′ phosphoramidate, resulting from the substitution of a ribosyl 3′-oxygen with an amine group. Further, the term nucleotide also includes those species that have a detectable label, such as for example a radioactive or fluorescent moiety, or mass label attached to the nucleotide.

The term “polynucleotide” refers to polymers of two or more nucleotides, and includes, but is not limited to, DNA, RNA, DNA/RNA hybrids including polynucleotide chains of regularly and/or irregularly alternating deoxyribosyl moieties and ribosyl moieties (i.e., wherein alternate nucleotide units have an —OH, then and —H, then an—OH, then an—H, and so on at the 2′ position of a sugar moiety), and modifications of these kinds of polynucleotides, wherein the attachment of various entities or moieties to the nucleotide units at any position are included.

The term “ribonucleotide” and the term “ribonucleic acid” (RNA), refer to a modified or unmodified nucleotide or polynucleotide comprising at least one ribonucleotide unit. A ribonucleotide unit comprises an hydroxyl group attached to the 2′ position of a ribosyl moiety that has a nitrogenous base attached in N-glycosidic linkage at the 1′ position of a ribosyl moiety, and a moiety that either allows for linkage to another nucleotide or precludes linkage.

The term “RNA interference” and the term “RNAi” are synonymous and refer to the process by which a polynucleotide (a miRNA or siRNA) comprising at least one polyribonucleotide unit exerts an effect on a biological process. The process includes, but is not limited to, gene silencing by degrading mRNA, attenuating translation, interactions with tRNA, rRNA, hnRNA, cDNA and genomic DNA, as well as methylation of DNA with ancillary proteins.

The term “siRNA” and the phrase “short interfering RNA” refer to unimolecular nucleic acids and to nucleic acids comprised of two separate strands that are capable of performing RNAi and that have a duplex region that is between 14 and 30 base pairs in length. Additionally, the term siRNA and the phrase “short interfering RNA” include nucleic acids that also contain moieties other than ribonucleotide moieties, including, but not limited to, modified nucleotides, modified internucleotide linkages, non-nucleotides, deoxynucleotides and analogs of the aforementioned nucleotides.

siRNAs can be duplexes, and can also comprise short hairpin RNAs, RNAs with loops as long as, for example, 4 to 23 or more nucleotides, RNAs with stem loop bulges, micro-RNAs, and short temporal RNAs. RNAs having loops or hairpin loops can include structures where the loops are connected to the stem by linkers such as flexible linkers. Flexible linkers can be comprised of a wide variety of chemical structures, as long as they are of sufficient length and materials to enable effective intramolecular hybridization of the stem elements. Typically, the length to be spanned is at least about 10-24 atoms. When the siRNAs are hairpins, the sense strand and antisense strand are part of one longer molecule.

Detailed descriptions of the criteria for the rational design of siRNA antisense strands for efficient gene silencing can be found in WO 2004/045543, WO 2006/006948, WO 2005/078095, WO 2005/097992, and WO 2005/090606, each of which are incorporated herein by reference in their entirety.

siRNAs can target any sequence including protein encoding sequences (e.g., open reading frames, ORFs), and non-coding sequences (e.g., 3′ UTRs, 5′ UTRs, intronic regions, promoter regions, microRNAs, piRNAs, enhancer regions, repetitive sequences, and more). In contrast, microRNA and piRNA mimics of the disclosure generally target a subset of genes and tools for predicting miRNA targets can be found in any number of publications including but not limited to Griffith-Jones, S. et al., Nucleic Acids Research, 2007.

The term “piRNAs” refers to Piwi-interacting RNAs, a class of small RNAs that are believed to be involved in transcriptional silencing (see Lau, N. C. et al (2006) Science, 313:305-306).

Correlations exist between miRNA expression patterns and key steps in mammalian development (Collignon, J. 2007 Dev Cell. 13(4):458-60; Conrad, R. et al., Birth Defects Res C Embryo Today. 2006 78(2):107-17). In addition, for a small collection of disorders, disease etiology has been correlated with mutations and/or mis-expression of specific miRNAs (Soifer, H. S. et al. 2007 Mol. Ther. 15:2070-2079; Clop, A., et al., 2006. Nat. Genet. 38:813-8). Still, neither the identity nor the role of all (human) miRNAs has been determined, leaving opportunities for new discoveries that have a significant impact on public health.

The present disclosure is based on the discovery that certain miRNAs exhibit differential expression levels in heart tissue from a mouse model of Familial Hypertrophic Cardiomyopathy (FHC) relative to normal heart tissue. Thus, some miRNAs are upregulated in diseased heart tissue, whereas others are down regulated in diseased tissue. See Table 1.

In one aspect, the disclosure provides miRNAs (along with their corresponding pri-miRNAs and pre-miRNAs) that are associated with hypertrophic cardiomyopathy and, accordingly, may be used as diagnostic and prognostic markers for heart disease. The miRNAs of the disclosure are provided in Table 1 which discloses both mouse miRNA and human miRNAs. In addition, the disclosure provides inhibitors and mimics of the aforementioned miRNAs that are useful as therapeutic agents for the treatment of heart disease, including the treatment of the symptoms of heart disease. Methods of designing miRNA mimics and miRNA inhibitors are well-known in the art.

The disclosure also provides the following miRNA-related sequences:
I. a nucleotide sequence that is a fragment of a miRNA of Table 1;
II. a nucleotide sequence complementary to a miRNA of Table 1 or to a nucleotide sequence of I;
III. a nucleotide sequence which has at least 80% identity to a miRNA of Table 1, or has at least 80% identity to a nucleotide sequence of I or II;
IV. a nucleotide sequence that hybridizes under stringent conditions to a miRNA of Table 1, or hybridizes under stringent conditions (see, e.g., Southern, 1975, J. Mol. Biol. 98:503-517) to a nucleotide sequence of I, II, or III.

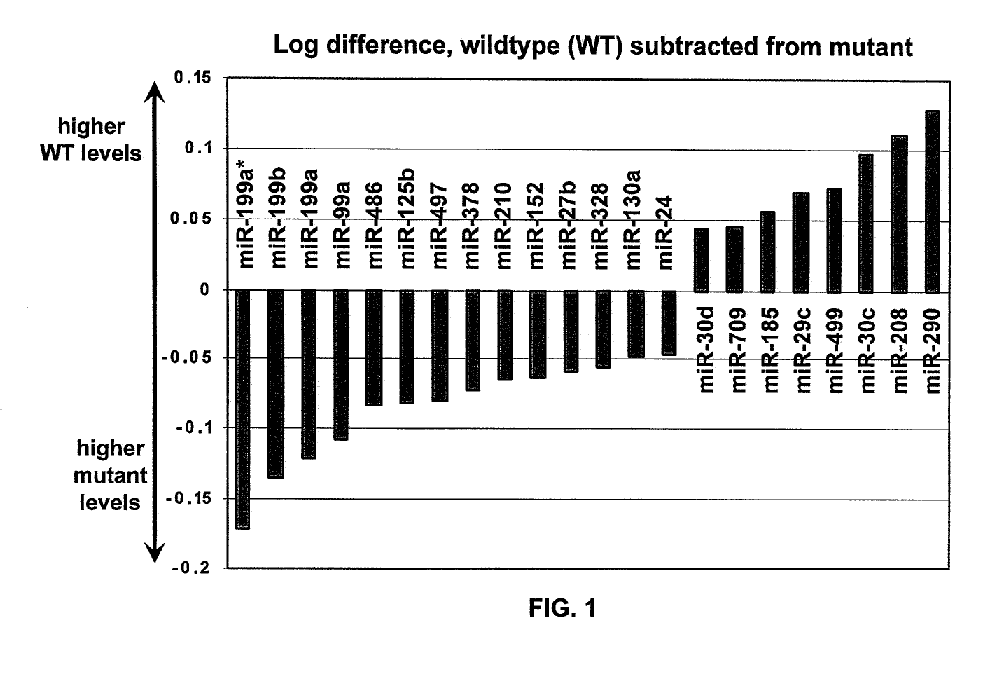

Changes in the expression levels of one or more miRNAs of the disclosure (and/or the level(s) of any corresponding pri-miRNA and/or pre-miRNA) is indicative of hypertrophic cardiomyopathy. See FIG. 1 and Table 1 which indicate that certain miRNAs are upregulated (e.g., overexpressed) whereas certain miRNAs are down-regulated (e.g., underexpressed) in diseased tissue in comparison to normal tissue. Thus, in one aspect, the disclosure provides a method of diagnosing FHC and related diseases of hypertrophic cardiomyopathy using the miRNAs described herein in Table 1, and/or for providing a prognosis (e.g., an estimate of disease outcome) for FHC and related diseases of hypertrophic cardiomyopathy using the miRNAs described herein in Table 1. In one embodiment, the method comprises the steps of 1) determining the expression level of one or more of the miRNAs of Table 1 in a heart sample from an individual suspected of having heart disease and 2) comparing the level of the one or more miRNAs with that observed in a normal individual known to not have heart disease, whereby diagnostic or prognostic information may be obtained.

In another embodiment, the method comprises the steps of 1) determining the expression level of one or more miRNA-related nucleotide sequences in a heart sample from an individual suspected of having heart disease, and 2) comparing the level of the one or more miRNA-related nucleotide sequences with that observed in a normal individual known to not have heart disease, whereby diagnostic or prognostic information may be obtained. In this embodiment, the one or more miRNA-related nucleotide sequences are, independently,
I. a nucleotide sequence that is a fragment of a miRNA of Table 1;
II. a nucleotide sequence complementary to a miRNA of Table 1 or to a nucleotide sequence of I;
III. a nucleotide sequence which has at least 80% identity to a miRNA of Table 1, or has at least 80% identity to a nucleotide sequence of I or II;
IV. a nucleotide sequence that hybridizes under stringent conditions to a miRNA of Table 1, or hybridizes under stringent conditions to a nucleotide sequence of I, II, or III.

The miRNAs or miRNA-related nucleotide sequences used as diagnostic or prognostic markers may be utilized individually or in combination with other molecular markers for heart disease, including without limitation other miRNAs, mRNAs, proteins, and nucleotide polymorphisms.

A range of techniques well known in the art can be used to quantitate amounts of one or more miRNAs or miRNA-related sequences of the disclosure from e.g., a biological sample. For instance, complements of the mature miRNA sequences of the disclosure can be associated with a solid support (e.g., a microarray) and purified RNA from e.g., clinical or control samples can be fluorescently labeled and profiled to determine whether the patient is suffering from FHC or related diseases of the heart (see Baskerville, S. et al. RNA 11:241-7). One preferred microarray platform is described in the document in PCT/US2007/003116, published as WO 2008/048342, which is incorporated herein by reference. Alternatively, quantitative PCR-based techniques (including real-time quantitative PCR techniques) can be used to assess the relative amounts of any of the miRNAs of the disclosure derived from e.g., control and/or test samples (Duncan, D. D. et al. 2006 Anal. Biochem. 359:268-70). In addition, Northern blotting, affinity matrices, in situ hybridization, and in situ PCR may be used. These techniques are all well known in the art.

Preferably, statistical methods are used to identify significant changes in miRNA levels for the aforementioned prognostic and diagnostic assays. For example, in one embodiment, p values are calculated using known methods to determine the significance in the change of the level of expression of a miRNA. In some embodiments, a value of p<0.05 is used as a threshold value for significance.

Samples for the prognostic and diagnostic assays of the disclosure may be obtained from an individual (e.g., a human or animal subject) suspected of having heart disease using any technique known in the art. For example, the sample may be obtained from an individual who is manifesting clinical symptoms that are consistent with the existence of heart disease, or from an individual with no clinical symptoms but with a predisposition towards developing heart disease due to genetic (e.g., a family history of heart disease and/or a known genetic predisposition towards heart disease) or environmental factors. Samples may be obtained by extracting a small portion of heart tissue from an individual using, for example, a biopsy needle.

In another aspect, the disclosure provides a method of treating FHC or related diseases of cardiac hypertrophy (e.g., ranging from at least partial relief of one or more symptoms to a complete cure) or preventing FHC or related diseases of cardiac hypertrophy by modulating the levels of a miRNA of Table 1. In some instances, the expression of miRNA sequences of the disclosure are down regulated in diseased tissues and re-introduction of one or more of these miRNAs may relieve or alleviate the symptoms of the disease. FIG. 1 demonstrates that the following miRNAs are down-regulated in diseased tissue: miR-30d, miR-709, miR-185, miR-29c, miR-499, miR-30c, miR-208, and miR-290. Thus, in one embodiment, the method for treating or preventing FHC or related diseases of cardiac hypertrophy comprises delivering one or more therapeutic miRNAs comprising the sequences of miR-30d, miR-709, miR-185, miR-29c, miR-499, miR-30c, miR-208, or miR-290 (or the human orthologs thereof) to individual in need thereof. Alternatively, or in addition, pri-miRNA, pre-miRNA, and/or miRNA mimics corresponding to these miRNAs of Table 1 may be employed in such methods of treatment. Such agents can be used individually, in combination with other miRNA mimics or inhibitors described herein, in combination with miRNAs mimics or inhibitors previously described, or in combination with other agents (e.g., small molecules such as beta blockers) used to treat FHC or related diseases.

In some instances, the expression of miRNA sequences of the disclosure are up regulated in diseased tissues and knockdown of one or more of these miRNAs may relieve or alleviate the symptoms of the disease. FIG. 1 demonstrates that the following miRNAs are up-regulated in diseased tissue: miR-199a*, miR-199b, miR-199a, miR-99a, miR-486, miR-125b, miR-497, miR-378, miR-210, miR-152, miR-27b, miR-328, miR-130a, and miR-24. Thus, in another embodiment, the method for treating or preventing FHC or related diseases of cardiac hypertrophy comprises delivering inhibitors of one or more of miR-199a*, miR-199b, miR-199a, miR-99a, miR-486, miR-125b, miR-497, miR-378, miR-210, miR-152, miR-27b, miR-328, miR-130a, or miR-24 (or an inhibitor of the human orthologs thereof) to an individual in need thereof. Such agents can be used individually, in combination with other miRNA mimics or miRNA inhibitors, in combination with miRNA mimics or inhibitors previously described, or in combination with other agents (e.g., small molecules) used to treat FHC or related diseases.

Synthetic, therapeutic miRNAs (microRNA mimics) or miRNA inhibitors of the miRNAs of Table 1 can be generated using a range of art-recognized techniques (e.g. ACE chemistry, see U.S. Pat. Nos. 6,111,086; 6,590,093; 5,889,136; and 6,008,400) and introduced into cells by any number of methods including electroporation-mediated delivery, lipid-mediated delivery, or conjugate-mediated delivery (including but not limited to cholesterol or peptide-mediated delivery). In still other instances, therapeutic miRNAs and inhibitors can be delivered using a vector (e.g., plasmid) or viral (e.g., lentiviral) expression system. One preferred expression system is described in US Provisional Patent Application No. 60/939,785, now published as WO 2008/147837. Studies have demonstrated that not all miRNAs are processed with equal efficiency. Thus, while it is possible to deliver a desired miRNA to a cell by expressing the related primary miRNA (pri-miRNA), in some instances it may be desirable to incorporate the mature sequence of the miRNA of the disclosure into a highly processed scaffold (e.g., miR-196a-2) would ensure efficient processing and expression.

Therapeutic miRNAs and inhibitors of the disclosure can contain modifications that enhance functionality, specificity, strand usage, and stability. For instance, 2′-O-methyl modifications, locked nucleic acids (LNAs), morpholinos, ethylene-bridged analogs (ENAs), 2′-O—F modifications, and phosphorothioate modifications can greatly enhance the stability of double stranded RNAs in serum. Similarly, addition of 2′-O-methyl modifications to positions 1 and 2 (counting from the 5′ end of the molecule) in the sense strand can enhance functionality and specificity (see U.S. patent application Ser. No. 11/019,831, published as U.S. Patent Application Publication No. 2005/0223427). Mimics and inhibitors can be delivered using an array of techniques including lipid mediated delivery, electroporation, and expression based systems (see, for instance, Ebert, M. S. et al. 2007 Nature Methods. 4: 721-6).

Pharmaceutical compositions comprising the inhibitors, mimics, siRNAs, and small molecules of the disclosure are also expressly contemplated and may be used for the treatment or prevention of FHC or related diseases of cardiac hypertrophy. Such pharmaceutical formulations preferably also comprise one or more pharmaceutically acceptable carriers or excipients, and may be formulated for a variety of routes of administration, including systemic and topical or localized administration. Techniques and formulations generally may be found in Remmington's Pharmaceutical Sciences, Meade Publishing Co., Easton, Pa. For systemic administration, injection is preferred, including intramuscular, intravenous, intraperitoneal, and subcutaneous. For injection, the therapeutic compositions of the invention can be formulated in liquid solutions, preferably in physiologically compatible buffers such as Hank's solution or Ringer's solution. In addition, the therapeutic compositions may be formulated in solid form and redissolved or suspended immediately prior to use. Lyophilized forms are also included. Preparations for oral administration are also contemplated, and may be formulated in a conventional manner to give either immediate or controlled release.

The pharmaceutical compositions of the disclosure may also include a second active ingredient for the treatment of heart disease e.g. a beta blocker. Additional active ingredients may also be added.

In addition to their use in vivo, the inhibitors, mimics, siRNAs, and small molecules of the disclosure can be delivered to cells ex vivo (e.g., to cells or tissues in culture) in order to modulate the level of an miRNA of Table 1. Thus, in another aspect the disclosure provides isolated cells and isolated tissues comprising the inhibitors, mimics, siRNAs, and small molecules of the disclosure. Synthetic mimics and inhibitors can be delivered to cells by a variety of methods including, but not limited to, lipid (e.g. DharmaFECT1, Thermo Fisher Scientific) or chemical (e.g. calcium phosphate) mediated transfection, electroporation, lipid-independent delivery via conjugation to one or more entities that mediate lipid- or chemical-independent delivery (e.g. conjugation of cholesterol), or any other method that has been identified or will be identified for nucleic acid transfer to target cells.

In addition, mimics and inhibitors can be delivered to a cell using an expression construct (e.g., based on a plasmid vector with appropriate cloning and promoter sequences) that expresses the sequence(s) that encode the miRNA mimic or miRNA inhibitor of choice. Such expression vectors can be introduced into cells (including cells within an organism such as a human being) by art-recognized transfection methods (e.g., Lipofectamine 2000, Invitrogen) or via viral-mediated delivery (e.g. lentiviral, adenoviral). Thus, in another aspect the disclosure provides expression constructs that direct expression of the mimics and inhibitors of the disclosure, and the disclosure further provides isolated cells and isolated tissues that comprise these expression constructs.

In another embodiment, the miRNAs described herein can be used as molecular markers in drug screening assays during drug development. Typically, in the early stages of drug development, in vitro studies involving cultured cells that often mimic one or more aspects of diseased tissue are performed to identify molecules that induce desirable phenotypes. As up or down regulation of miRNAs described herein are indicative of e.g. the FHC phenotype (or hypertrophic cardiomyopathy in general), they can be used as markers during drug development to identify agents that positively effect clinical outcomes. In one preferred example, one or more of the miRNAs described herein are used to screen a collection of small molecules to identify agents that modulate the expression of sequences listed in the enclosed tables. Agents that cause e.g., miRNA(s) expression levels to return to a level that is more normal would be considered potential therapeutic candidates.

In another example, one or more of the miRNAs described herein are used as prognostic indicators to judge the effectiveness of drug treatment regimes. For example, the levels of miRNAs of the disclosure can be assessed in FHC patients receiving a particular treatment to determine the effectiveness of the treatment in lessening one or more phenotypes of the disease.

The miRNAs of the disclosure can be identified as single stranded pri-miRNA or pre-miRNA hairpin structures (wherein a hairpin is defined as an oligonucleotide that is about 40-150 nucleotides in length and contains secondary structures that result in regions of duplex and loops) or characterized as mature double stranded miRNAs. The miRNAs are capable of entering the RNAi pathway, being processed by gene products associated with the pathway (e.g., Drosha, Dicer, and the RNA Interference Silencing Complex, RISC), and inhibiting gene expression by translation attenuation or message (mRNA) cleavage. As such, all of the miRNAs of the invention can be described by multiple labels depicting the level of processing. Furthermore, all of the miRNAs disclosed herein can be found at http://microrna.sanger.ac.uk/sequences/.

With respect to the sequence of pri-, pre-, and mature miRNA sequences, it is worth noting that the field of RNAi and thus the sequences and structures associated with human, mouse and rat miRNAs varies slightly as versions of the Sanger miRNA database (miRBase) evolve. Though these newer versions of e.g. miRbase can have sequences that are extended and/or truncated on either the 5′ or 3′ end of the mature and passenger sequences, the changes do not alter the overall identity of the miRNA nor the ability to utilize these sequences in the context of the described embodiments.

In another aspect the disclosure provides a list of genes that are differentially expressed in mutant murine heart tissues. See Table 2. The genes of Table 2 were identified according to the methods of the examples. The genes listed in Table 2 include genes that are directly modulated by the miRNAs of Table 1, as well as genes that are indirectly modulated by the miRNAs of Table 1. Direct or indirect modulation of the genes of Table 2 is likely indicative of and plays a role in induction of e.g. hypertrophic cardiomyopathy. In the context of this document, the term “direct modulation” indicates the messenger RNA of the target gene is acted upon directly by one or more miRNAs of the disclosure. These interactions are mediated by elements of the RNAi pathway including but not limited to RISC. “Indirect modulation” indicates expression of the target gene is altered as a result of events down-stream of direct interaction between an miRNA and a target gene. For example, a target gene may be indirectly modulated by a miRNA of Table 1 when that miRNA directly modulates the expression of an upstream gene, and the product of the upstream gene directly modulates the target gene.

We use the terms “miRNA target gene”, “miRNA target”, and “target gene” interchangeably herein to refer to genes that are directly modulated by specific miRNA(s). Extensive studies into the mechanism of miRNA action have identified characteristic features of miRNA target genes. These include the presence of the 3′ UTR target sites (e.g. seed complements), the number and positioning of seed complements within a 3′ UTR, preferences for local AU-rich sequences and more (see, for instance, Grimson, A. et al 2007. Mol Cell 27:91-105). As such, miRNA target genes can be identified bioinformatically (e.g., see the miRNA target prediction site at http://www.russell.embl-heidelberg.de/miRNAs/; Targetscan, http://www.targetscan.org/mamm_{—}31/), by microarray analysis (Huang et. al., 2007, Nature Methods 4:1045-9), and by biochemical methods (Karginov, F. V., 2007, PNAS 104:19291-6). Table 3 lists those genes of Table 2 that are predicted to be target genes of the miRNAs of Table 1, along with the specific miRNA(s) that is likely to directly modulate each of those genes. Accordingly, the miRNA target genes of Table 3 are directly modulated by the miRNAs of Table 1 in cardiac tissue. Note that the list of genes in Table 3 is likely only a subset of all of the target genes of the miRNAs of Table 1.

Human orthologs of the genes of Tables 2-3 are also included in the disclosure. The sequences of the human orthologs of the genes in Tables 2-3 may be obtained from well-known public sources using standard bioinformatics techniques that are routine in the art. Genes of the disclosure (e.g., Tables 2-3) are identified by NCBI Accession and GI (also referred to as “gi”) numbers. Full descriptions of these can be obtained at http://www.ncbi.nlm.nih.gov/.

Changes in the expression levels of one or more of the genes of Table 2 and Table 3 of the disclosure are indicative of hypertrophic cardiomyopathy. Thus, in another aspect the disclosure provides methods of diagnosing and/or providing a prognosis (e.g., an estimate of disease outcome) for FHC and related diseases of hypertrophic cardiomyopathy by measuring the level of expression of one or more of the genes of Tables 2-3. In one embodiment, a method of diagnosing, and/or providing a prognosis for FHC and related diseases of hypertrophic cardiomyopathy comprises 1) determining the expression level of one or more of the genes of Tables 2-3 in a heart sample from (e.g. a patient) and 2) comparing the expression level of the one or more of the genes of Tables 2-3 with that observed in normal patients. Preferably, the gene whose expression level is determined is a miRNA target gene from Table 3, including a human ortholog of any gene from Table 3. The genes of Tables 2-3 may be used as diagnostic markers either individually or in combination with other molecular markers disclosed herein or identified in previous or future studies (e.g., other miRNAs, proteins, nucleotide polymorphisms).

With respect to the diagnostic value of the genes disclosed herein, a range of techniques known in the art can be used to quantitate the expression level of the genes of the disclosure from e.g., a biological sample. In some embodiments, the mRNA produced by a gene from Tables 2-3 is measured using, for example, PCR-based methods (e.g., quantitative RT-PCR), microrray-based methods, Northern blotting, or any other technique known in the art for measuring the level of mRNA (see also the methods disclosed above for measuring miRNA levels which are generally applicable to the measurement of mRNA also). In other embodiments, the level of the protein encoded by a gene from Tables 2-3 is measured using, for example, western blots, antibody arrays, ELISA assays, or any other technique known in the art.

In another aspect, the disclosure provides a method of treating or preventing FHC or related diseases of cardiac hypertrophy by modulating the levels of one or more genes from Tables 2-3. In some instances, one or more genes from Tables 2-3 are down regulated in diseased tissues as a result of up-regulation of one or more of the miRNAs of Table 1. Thus, modulation of these genes may relieve or alleviate the symptoms of the disease. In one embodiment, a method of treating or preventing FHC or related diseases of cardiac hypertrophy comprises increasing the expression level of one or more of the genes of Tables 2-3 (e.g., increasing the level of transcription and/or translation) or increasing the activity level of the protein product of one or more of the genes of Tables 2-3 (e.g., increasing the activity of a protein encoded by a target gene). In one preferred method, the modulation of one or more of the genes of Tables 2-3 can be achieved by altering the levels of the miRNA(s) that target that gene. Preferably, the gene that is modulated is a miRNA target gene from Table 3, including a human ortholog of any gene from Table 3.

In another instance, one or more of the genes from Tables 2-3 are up-regulated in diseased tissues as a result of down-regulation of one or more of the miRNAs of Table 1, and suppression of the function of said genes may relieve or alleviate the symptoms of the disease. Thus, another embodiment a method of treating or preventing FHC or related diseases of cardiac hypertrophy comprises suppressing one or more of the genes of Tables 2-3. Suppression of gene function can be achieved by a wide range of methods including gene knockdown using siRNA or antisense molecules, the use of small molecule inhibitor of, for example, protein function, the use of neutralizing antibodies against the protein encoded by the gene, or other means. Alternatively, suppression of genes can be achieved by increasing the concentration of one or more miRNAs that target that gene. Preferably, the gene is a miRNA target gene from Table 3 which is indicated as having increased expression in mutant heart tissue (see column 2 of Table 3 for an indication of the expression level in mutant heart tissue in comparison to wild-type heart tissue).

The aforementioned methods of treating or preventing FHC or related diseases of cardiac hypertrophy are carried out in an individual (e.g., a human patient) in need of such treatment or prevention. Pharmaceutical compositions suitable for such methods may be formulated in accordance with the disclosure above concerning the formulation of pharmaceutical compositions comprising miRNA mimics, inhibitors etc. In addition, the modulation of the genes of Tables 2-3 (either an increase or decrease) can be carried out ex vivo e.g., using cells or tissue in vitro.

In another embodiment, the genes of Tables 2-3 can be used as molecular markers in drug screening assays during drug development. Typically, in the early stages of drug development, in vitro studies involving cultured cells that often mimic one or more aspects of diseased tissue are performed to identify molecules that induce desirable phenotypes. As up or down regulation of the genes of Tables 2-3 are indicative of e.g. the FHC phenotype, they can be used as markers during drug development to identify agents that positively effect clinical outcomes. In one preferred example, one or more of the genes of Tables 2-3 are used to screen a collection of small molecules to identify agents that modulate their expression. Agents that cause e.g., gene expression levels to return to a level that is more normal would be considered potential therapeutic candidates.

In another example, one or more of the genes of Tables 2-3 are used as prognostic indicators to judge the effectiveness of drug treatment regimes. For example, the levels of expression of one or more of the genes of Tables 2-3 can be assessed in FHC patients receiving a particular treatment to determine the effectiveness of the treatment in lessening one or more phenotypes of the disease.

Evolutionarily, microRNAs are highly conserved sequences. For instance, while the sequences flanking the mature sequence of e.g., miR-let-7C differ from species to species, the mature sequences themselves and the targets are heavily conserved. For this reason, though the described studies were performed on rodents carrying mutations in the myosin heavy chain, it is predicted that human patients with mutations in 1) the human myosin heavy chain gene, or 2) carrying mutations in other subunits of the sarcomere that similarly effect sarcomere function, or 3) have mutations in other genes that similarly effect sarcomere function, will also exhibit similar sets of miRNAs and miRNA target perturbations. Furthermore, since there are multiple diseases that can induce hypertrophy in the heart, there is a high likelihood that the miRNAs and genes identified here will show similar modulation in non-FHC diseases that also exhibit cardiac hypertrophy or possibly other forms of cardiac dysfunction, such as hypertensive heart disease or myocardial infarction (heart attack).

The following examples are non-limiting are provided solely to aid in the understanding of the disclosure.

### EXAMPLES

### Example 1

### Identification of miRNAs Associated with Mice Carrying Mutations in the Myosin Heavy Chain Gene

MicroRNAs are widely expressed in heart tissue and there are miRNAs that may be specific and/or important to the heart either in expression patterns or clinical importance. A study of hypertrophic cardiomyopathy was performed by investigating a mouse model that carries a point mutation (Arg403Gln) and a deletion (AA468-527) in the gene encoding the myosin heavy chain. As 1) the mutations carried by these animals are similar to those observed in humans that exhibit Familial Hypertrophic Cardiomyopathy (FHC), and 2) mutant mice exhibit many of the phenotypes observed in FHC patients, this approach closely mimics conditions observed in inflicted humans and therefore represents a powerful tool for identifying therapeutic targets and prognostic/diagnostic molecular markers of human FHC and other diseases that induce similar phenotypic characteristics.

A study of cardiac miRNA expression patterns has been performed in the described genetic mouse model using a novel miRNA microarray platform which is highly sensitive and allows accurate, side-by-side comparisons of miRNA profiles of tissue samples taken from e.g., different animals (see PCT/US2007/003116). As these studies have 1) been performed with murine models that contain mutations similar to those observed in human systems, and 2) were performed on aged mice, they accurately define the set of circumstances observed in human patients suffering variants of hypertrophic cardiomyopathy such as FHC.

To identify miRNAs that are associated with FHC, hearts from normal and mutant male mice (8 month old mice carrying, 1) the Arg403Gln point mutation, and 2) a deletion of AA468-527, in the myosin heavy chain gene (see Vikstrom et al., 1996, Molecular Medicine 2:556-567) were collected and homogenized. RNA from normal and mutant samples was purified (Trizol preparations, Invitrogen) and then labeled. Specifically, 200 ng of mouse total RNA was dephosphorylated with calf intestinal phosphatase, to reduce intramolecular ligation. pCp-DY649 was ligated to the 3′ end of RNA molecules with T4 RNA ligase. The excess dye was removed by passing the ligation reactions through a size-exclusion column. The column flow-through contained the labeled microRNAs,

Labeled samples were then mixed with a previously designed reference library containing Cy3-analog labeled oligonucleotides for each of the mouse miRNAs (Dharmacon) and hybridized for 20 hours (53° C.) to microarray chips containing probes for all of the murine miRNAs (sequences based on mirBase 9.0, probe design based on PCT/US2007/003116). Arrays were then washed and scanned on an Agilent G2565 microarray scanner.

Analysis of the microarray data was performed to identify miRNAs that were associated with the disease state. To accomplish this, the average fluorescence value of each technical and biological replicate of each miRNA was first determined using the Agilent Feature Extraction software. The relative signal intensity and error modeling value for each microRNA sequence was then generated using a data analysis software program developed in-house. Results were then imported in the Rosetta Resolver biosoftware (Rosetta Inpharmatics) for higher order analysis and an analysis of variance (ANOVA) was performed on the dataset to generate a list of significantly differentially expressed microRNAs (p-value cutoff of 0.05). Output of these experiments was based on six biological replicates (different mice) and three technical replicates (different arrays) representing each genotype used in these studies.

Results from these studies identified twenty-two murine miRNAs that were observed to be up- or down-regulated in mice carrying the Arg403Gln point and AA468-527 deletion mutations. A list of the miRNAs identified by this study is provided in Table 1 along with the human counterparts (orthologs). Table 1 lists both Sanger miRBase 9.0 and Sanger miRBase 10.1 names for the identified mouse and human miRNAs. In addition, a plot of the log difference of wild-type and mutant miRNA levels of validated miRNAs is presented in FIG. 1.

Hierarchal clustering was performed to determine whether the modulation of the miRNAs of the disclosure correlated with the disease. Specifically, heatmaps and hierarchal clustering data were generated for mutant and wild type tissues using 1) a random group of 22 miRNAs, and 2) the miRNAs identified in Table 1. A heatmap is a graphical representation of relative intensity values for each miRNA. Relative intensities determined from the microRNA microarray were subjected to Z-score transformation which adjusts the intensity values such that the mean for the measurement of each miRNA across the samples is zero with a standard deviation of one. Therefore, the relative value of each miRNA across the samples becomes more apparent after Z-score transformation. Agglomerative clustering analysis performed on the randomly-chosen set of 22 microRNAs failed to segregate animals into wild type and mutant groups i.e. failed to segregate animals on the basis of genotype/phenotype, suggesting this collection of randomly-selected miRNAs are not associated with the disease. In contrast, when the cluster analysis is performed with the miRNAs described in Table 1, five of the six mutant samples cluster together, indicating that a correlation exists between the expression pattern of the disclosed miRNAs and the disease phenotype.

### Example 2

### Identification of Genes by Microarray Analysis and Bioinformatic Methods

Whole genome microarrary profiling was used in conjunction with seed-based bioinformatic selection techniques to identify miRNA target genes for the miRNAs of the disclosure. Specifically, total RNA from wild type and mutant murine heart tissues was isolated and labeled with Cy5. Subsequently, equal amounts of each sample were mixed with a Cy3-labeled universal mRNA sample (Stratagene) and hybridized to Agilent's mouse whole genome dual mode expression array (Agilent Technologies, Santa Clara, Calif.) according to the manufacturer's recommendations. Following hybridization, arrays were washed according to manufacturers instructions, scanned (Agilent G2565 microarray scanner), and then assessed to identify genes that were differentially expressed in mutant and wild type tissues.

Table 2 provides a list of the genes (identified by Accession number, GI number and gene name) that are differentially expressed in mutant and wild type tissues. For each gene, Table 2 also provides the Log (ratio) of mutant and wild type (WT) signal with respect to the Stratagene Universal Mouse Reference, and the log difference (Diff) calculated by subtracting the mutant Log (ratio) from the wild type Log (ratio). The log (ratio) is a value that represents relative expression of a gene compared to the universal reference sample used in these studies. Comparison of each of the samples (mutant and wild type) against the universal sample then allows accurate comparison of the levels of each transcript in the mutant and wild type samples. The values shown in the table for Log (ratio) for wild type and mutant are the average values for the biological and technical replicates for each genotype. The log difference calculates the difference in expression between wild type and mutant samples. Thus, for example, the gene having Accession number NM_{—}009349 (bolded in Table 2) has a Log Diff of −0.40987 which is equivalent to a 2.57-fold up-regulation in the mutant tissue compared to wild type. Table 2 also lists whether a particular gene is increased or decreased in expression in the mutant heart tissue relative to wild type heart tissue (based on the Log Diff).

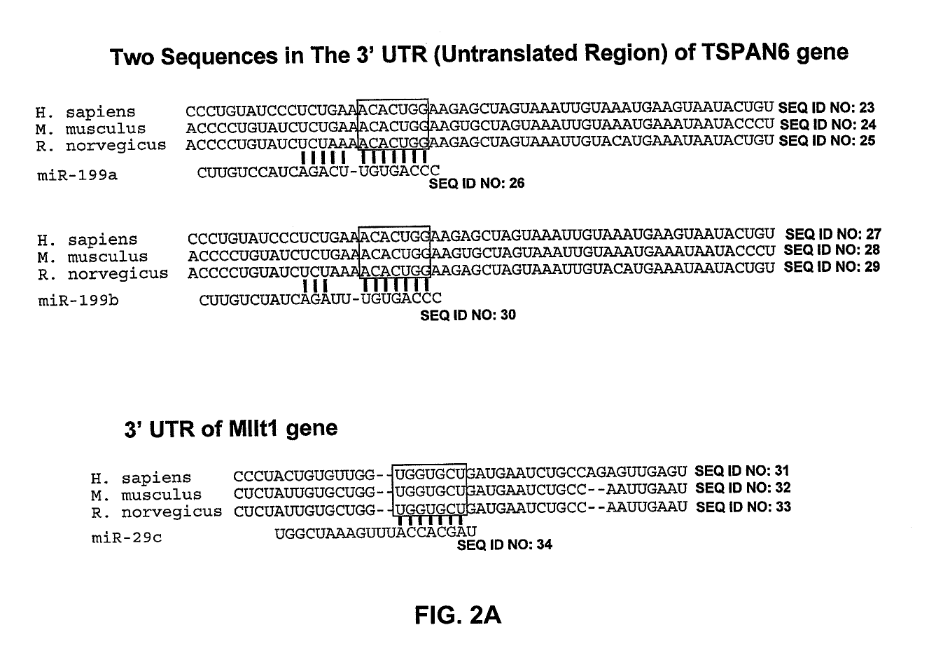

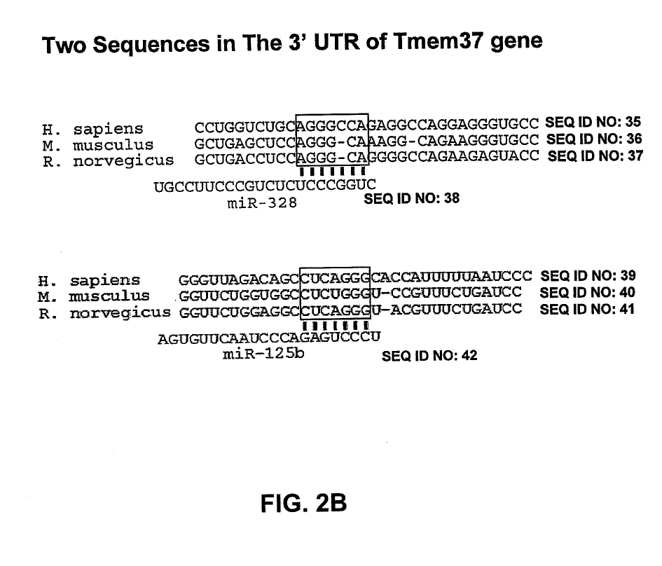

Table 2 represents a list of genes that may be directly-modulated by the miRNAs listed in Table 1 (i.e., some or all of the genes of Table 2 are likely to be miRNA target genes for the miRNAs of Table 1). To identify a list of miRNA target genes which are likely to interact directly with the miRNAs identified in Table 1, the sequence of the 3′ UTR of each of the genes identified in Table 2 were scanned (using TargetScan 4.0, available from http://www.targetscan.org/) to bioinformatically identify genes that contained seed complements to one or more of the miRNAs identified in Table 1. Previous studies have identified 3′ UTR seed complements as being important in miRNA targeting (see, for instance, Birmingham et al, 2006, Nature Methods 3(3):199-204). The genes identified using this bioinformatic approach are listed in Table 3. Thus, Table 3 provides the identity of genes that 1) are differentially modulated in mutant and wild type tissues and 2) contain one or more seed matches to the miRNAs identified in Table 1. Accordingly, the genes in Table 3 may be target genes (i.e., directly-modulated) for the miRNAs of the disclosure (see Table 1). Table 3 provides the mouse gene name, the miRs which are predicted to target the gene, the gene name for the human ortholog, and the GI number for the human ortholog. FIG. 2A-2B provides an example of miR-199a, miR-199b, miR-29c, and miR-328 alignment with 3′ UTR target sites identified bioinformatically.

TABLE 1

| | | | | | Expression |
|---|---|---|---|---|---|
| | | | | | Mutant Level |
| | Mouse | | Human | | in Heart |
| | miRNA | Human | Ortholog | | Tissue |
| Mouse miRNA | Ver 10.1 | Ortholog | Ver 10.1 | Mouse Stem-Loop Sequence (5′-3′) | (compared to |
| Ver 9 name | Name | Ver 9 Name | Name | (Mature Sequence Underlined) | wild-type) |
| mmu-miR-378 | same | hsa-miR-378 | hsa-miR-378 | AGGGCUCCUGACUCCAGGUCCUGUGUGUUACCU | Increased |
| | | | | CGAAAUAGCACUGGACUUGGAGUCAGAAGGCCU | |
| | | | | (SEQ ID NO: 1) | |
| mmu-miR-99a | same | hsa-miR-99a | hsa-miR-99a | CAUAAACCCGUAGAUCCGAUCUUGUGGUGAAGU | Increased |
| | | | | GGACCGCGCAAGCUCGUUUCUAUGGGUCUGUG | |
| | | | | (SEQ ID NO: 2) | |
| mmu-miR-125b | mmu-125b-5p | hsa-miR- | hsa-miR-125b | UGCGCUCCCCUCAGUCCCUGAGACCCUAACUUG | Increased |
| | | 125b(−1) | | UGAUGUUUACCGUUUAAAUCCACGGGUUAGGCU | |
| | | | | CUUGGGAGCUG | |
| | | | | (SEQ ID NO: 3) | |
| mmu-miR-199a* | mmu-miR-199a-3p | hsa-miR-199a | hsa-miR- | UGGAAGCUUCAGGAGAUCCUGCUCCGUCGCCCC | Increased |
| | | | 199a-3p | AGUGUUCAGACUACCUGUUCAGGACAAUGCCGU | |
| | | | | UGUACAGUAGUCUGCACAUUGGUUAGACUGGGC | |
| | | | | AAGGGCCAGCA | |
| | | | | (SEQ ID NO: 4) | |
| mmu-miR-199b | mmu-miR-199b | hsa-miR-199b | hsa-miR-199b | UGGAAGCUUCAGGAGAUCCUGCUCCGUCGCCCC | Increased |
| | | | | AGUGUUCAGACUACCUGUUCAGGACAAUGCCGU | |
| | | | | UGUACAGUAGUCUGCACAUUGGUUAGACUGGGC | |
| | | | | AAGGGCCAGCA | |
| | | | | (SEQ ID NO: 5) | |
| mmu-miR-486 | same | hsa-miR-486 | hsa-miR-486 | CAGCCAGCUCUGAUCUCGCCCUCCCUGAGGGGU | Increased |
| | | | | CCUGUACUGAGCUGCCCCGAGGUCCUUCACUGU | |
| | | | | GCUCAGCUCGGGGCAGCUCAGUACAGGAUGCGU | |
| | | | | CAGGGUGGGAGACAACGGGGAACAAGCCA | |
| | | | | (SEQ ID NO: 6) | |
| mmu-miR-497 | same | hsa-miR-497 | hsa-miR-497 | CCUGCCCCCGCCCCAGCAGCACACUGUGGUUUG | Increased |
| | | | | UACGGCACUGUGGCCACGUCCAAACCACACUGU | |
| | | | | GGUGUUAGAGCGAGGGUA | |
| | | | | (SEQ ID NO: 7) | |
| mmu-miR-328 | same | hsa-miR-328 | hsa-miR-328 | CUGUCUCGGAGCCUGGGGCAGGGGGGCAGGAGG | Increased |
| | | | | GGCUCAGGGAGAAAGUAUCUACAGCCCCUGGCC | |
| | | | | CUCUCUGCCCUUCCGUCCCCUGUCCCCAAGU | |
| | | | | (SEQ ID NO: 8) | |
| mmu-miR-208 | mmu-miR-208a | hsa-miR-208 | hsa-miR-208a | UUCCUUUGACGGGUGAGCUUUUGGCCCGGGUUA | Decreased |
| | | | | UACCUGACACUCACGUAUAAGACGAGCAAAAAG | |
| | | | | CUUGUUGGUCAGAGGAG | |
| | | | | (SEQ ID NO: 9) | |
| mmu-miR-210 | same | hsa-miR-210 | hsa-miR-210 | CCGGGGCAGUCCCUCCAGGCUCAGGACAGCCAC | Increased |
| | | | | UGCCCACCGCACACUGCGUUGCUCCGGACCCAC | |
| | | | | UGUGCGUGUGACAGCGGCUGAUCUGUCCCUGGG | |
| | | | | CAGCGCGAACC | |
| | | | | (SEQ ID NO: 10) | |
| mmu-miR-185 | same | hsa-miR-185 | hsa-miR-185 | AGGGAUUGGAGAGAAAGGCAGUUCCUGAUGGUC | Decreased |
| | | | | CCCUCCCAGGGGCUGGCUUUCCUCUGGUCCUU | |
| | | | | (SEQ ID NO: 11) | |
| mmu-miR-30d | same | hsa-miR-30d | hsa-miR-30d | AAGUCUGUGUCUGUAAACAUCCCCGACUGGAAG | Decreased |
| | | | | CUGUAAGCCACAGCCAAGCUUUCAGUCAGAUGU | |
| | | | | UUGCUGCUACUGGCUC | |
| | | | | (SEQ ID NO: 12) | |
| mmu-miR-24 | same | hsa-miR-24 | hsa-miR-24 | CUCCGGUGCCUACUGAGCUGAUAUCAGUUCUCA | Increased |
| | | | | UUUCACACACUGGCUCAGUUCAGCAGGAACAGG | |
| | | | | AG | |
| | | | | (SEQ ID NO: 13) | |
| mmu-miR-30c | same | hsa-miR-30c | hsa-miR-30c | ACCAUGUUGUAGUGUGUGUAAACAUCCUACACU | Decreased |
| | | | | CUCAGCUGUGAGCUCAAGGUGGCUGGGAGAGGG | |
| | | | | UUGUUUACUCCUUCUGCCAUGGA | |
| | | | | (SEQ ID NO: 14) | |
| mmu-miR-499 | same | hsa-miR-499 | hsa-miR-499 | GGGUGGGCAGCUGUUAAGACUUGCAGUGAUGUU | Decreased |
| | | | | UAGCUCCUCUGCAUGUGAACAUCACAGCAAGUC | |
| | | | | UGUGCUGCUGCCU | |
| | | | | (SEQ ID NO: 15) | |
| mmu-miR-29c | same | hsa-miR-29c | hsa-miR-29c | AUCUCUUACACAGGCUGACCGAUUUCUCCUGGU | Decreased |
| | | | | GUUCAGAGUCUGUUUUUGUCUAGCACCAUUUGA | |
| | | | | AAUCGGUUAUGAUGUAGGGGGA | |
| | | | | (SEQ ID NO: 16) | |
| mmu-miR-130a | same | hsa-miR-130a | hsa-miR- | GAGCUCUUUUCACAUUGUGCUACUGUCUAACGU | Increased |
| | | | 130a | GUACCGAGCAGUGCAAUGUUAAAAGGGCAUC | |
| | | | | (SEQ ID NO: 17) | |
| mmu-miR-290 | mmu-miR-290 | no exact hsa | no exact | CUCAUCUUGCGGUACUCAAACUAUGGGGGCACU | Decreased |
| | | match | hsa match | UUUUUUUUUCUUUAAAAAGUGCCGCCUAGUUUU | |
| | | | (but hsa- | AAGCCCCGCCGGUUGAG | |
| | | | miR-371 is | (SEQ ID NO: 18) | |
| | | | close | | |
| | | | match) | | |
| mmu-miR-27b | same | hsa-miR-27b | hsa-miR-27b | AGGUGCAGAGCUUAGCUGAUUGGUGAACAGUGA | Increased |
| | | | | UUGGUUUCCGCUUUGUUCACAGUGGCUAAGUUC | |
| | | | | UGCACCU | |
| | | | | (SEQ ID NO: 19) | |
| mmu-miR-199a | mmu-miR-199a-5p | hsa-miR- | hsa-miR- | UGGAAGCUUCAGGAGAUCCUGCUCCGUCGCCCC | Increased |
| | | 199a(−5p) | 199a-5p | AGUGUUCAGACUACCUGUUCAGGACAAUGCCGU | |
| | | | | UGUACAGUAGUCUGCACAUUGGUUAGACUGGGC | |
| | | | | AAGGGCCAGCA | |
| | | | | (SEQ ID NO: 20) | |
| mmu-miR-709 | same | no hsa | no hsa | UGUCCCGUUUCUCUGCUUCUACUCAGAAGUGCU | Decreased |
| | | counterpart | counterpart | CUGAGCAUAGAACUGUCCUGUUUGAGCAGCACU | |
| | | | | GGGGAGGCAGAGGCAGGAGGAU | |
| | | | | (SEQ ID NO: 21) | |
| mmu-miR-152 | same | hsa-miR-152 | hsa-miR-152 | CCGGGCCUAGGUUCUGUGAUACACUCCGACUCG | Increased |
| | | | | GGCUCUGGAGCAGUCAGUGCAUGACAGAACUUG | |
| | | | | GGCCCGG | |
| | | | | (SEQ ID NO: 22) | |

TABLE 2

| | | | Log Diff | | | Expression |
|---|---|---|---|---|---|---|
| Gene | Log | Log | (WT | | | in mutant |
| Accession | (Ratio) | (Ratio) | minus | Gene Name | | compared to |
| No: | [WT] | [Mutant] | mutant] | (Mouse) | GI No: | WT |
| AK002420 | 1.06401 | 1.14816 | −0.08415 | 0610009L18Rik | 12832390 | Increased |
| NM_144846 | 0.09232 | −0.1333 | 0.22562 | 0910001A06Rik | 146149091 | Decreased |
| AK003900 | 0.03618 | −0.04898 | 0.08516 | 1110021J02Rik | 12834846 | Decreased |
| AK004470 | 0.22453 | 0.12024 | 0.10429 | 1190003J15Rik | 12835667 | Decreased |
| AK033642 | 0.75005 | 0.61251 | 0.13754 | 1300010F03Rik | 26329330 | Decreased |
| NM_030087 | −0.15248 | 0.00511 | −0.15759 | 1500032D16Rik | 140972308 | Increased |
| NM_024260 | −0.10837 | −0.20312 | 0.09475 | 1700034M03Rik | 142353064 | Decreased |
| NM_145216 | 0.32086 | 0.14616 | 0.1747 | 2210403B10Rik | 88853579 | Decreased |
| BC048160 | 0.0037 | 0.23932 | −0.23562 | 2210418O10Rik | 28878985 | Increased |
| NM_026279 | 0.72079 | 0.54409 | 0.1767 | 2310026E23Rik | 118131214 | Decreased |
| AK013475 | 0.39719 | 0.30089 | 0.0963 | 2310061C15Rik | 12850851 | Decreased |
| NM_025879 | 0.10308 | 0.03338 | 0.0697 | 2410002O22Rik | 148276982 | Decreased |
| BC065697 | −0.06059 | −0.13542 | 0.07483 | 2600014C01Rik | 41351298 | Decreased |
| NM_027263 | −0.95453 | −0.77956 | −0.17497 | 2610040C18Rik | 21312473 | Increased |
| NM_178112 | −0.27568 | −0.35193 | 0.07625 | 2810013E07Rik | 142344002 | Decreased |
| AK012940 | −0.00675 | 0.14638 | −0.15313 | 2810050O03Rik | 12850005 | Increased |
| NM_023320 | 0.41312 | 0.5429 | −0.12978 | 2810052M02Rik | 118129883 | Increased |
| NM_026038 | 0.0862 | −0.06267 | 0.14887 | 2810055F11Rik | 141803478 | Decreased |
| NM_197980 | 0.34729 | 0.43513 | −0.08784 | 2810437L13Rik | 37574049 | Increased |
| BC046911 | 0.0864 | −0.11253 | 0.19893 | 4833442J19Rik | 28422332 | Decreased |
| NM_177101 | 0.10969 | −0.06787 | 0.17756 | 4833442J19Rik | 133892656 | Decreased |
| AK077044 | 0 | 0.1866 | −0.1866 | 4921501E09Rik | 26097176 | Increased |
| AK129150 | 0.17674 | 0.25444 | −0.0777 | 4930535B03Rik | 37359963 | Increased |
| BC025885 | −0.29703 | −0.21998 | −0.07705 | 4933439C20Rik | 22169842 | Increased |
| AK122299 | 0.34319 | 0.46461 | −0.12142 | 6430548M08Rik | 28972254 | Increased |
| AK078681 | 0.1264 | 0.20555 | −0.07915 | 7530404M11Rik | 26098037 | Increased |
| AK033113 | 0.27642 | 0.38464 | −0.10822 | 8030431J09Rik | 26083223 | Increased |
| NM_027829 | −0.02534 | 0.06935 | −0.09469 | 9030607L17Rik | 89886472 | Increased |
| AK018112 | 0.02495 | −0.31384 | 0.33879 | 9930013L23Rik | 12857678 | Decreased |
| AK087668 | −0.39772 | −0.26076 | −0.13696 | A330103N21Rik | 26104430 | Increased |
| NM_172510 | 0.1017 | −0.20088 | 0.30258 | A930031D07Rik | 167900453 | Decreased |
| NM_177470 | 1.24512 | 1.12399 | 0.12113 | Acaa2 | 142366266 | Decreased |
| NM_009606 | 1.54214 | 1.68844 | −0.1463 | Acta1 | 133893192 | Increased |
| NM_183274 | −0.12726 | −0.01941 | −0.10785 | Acta2 | 34304068 | Increased |
| NM_019785 | 0.04413 | −0.01628 | 0.06041 | Actr10 | 9789886 | Decreased |
| NM_134079 | 0.34482 | 0.17735 | 0.16747 | Adk | 146149178 | Decreased |
| AK019479 | −0.0918 | 0.06794 | −0.15974 | AK019479 | 12859711 | Increased |
| AK036850 | −0.05648 | 0.22282 | −0.2793 | AK036850 | 26085459 | Increased |
| AK047189 | −0.11359 | 0 | −0.11359 | AK047189 | 26092001 | Increased |
| AK079391 | 0.0373 | 0.12553 | −0.08823 | AK079391 | 26098473 | Increased |
| AK085108 | 0.42178 | 0.53574 | −0.11396 | AK085108 | 26102464 | Increased |
| NM_144903 | 0.95451 | 0.40467 | 0.54984 | Aldob | 31981737 | Decreased |
| NM_008537 | 0.44716 | 0.30242 | 0.14474 | Amacr | 142381661 | Decreased |
| NM_007446 | 1.19781 | 0.75909 | 0.43872 | Amy1 | 160358818 | Decreased |
| NM_146011 | −0.08072 | −0.19948 | 0.11876 | Arhgap9 | 90093350 | Decreased |
| AK028642 | 0.31239 | 0.44008 | −0.12769 | Arhgef6 | 26080958 | Increased |
| BC054531 | 0.25923 | 0.16351 | 0.09572 | Atp2a2 | 32452027 | Decreased |
| NM_016755 | 1.04584 | 0.94404 | 0.1018 | Atp5j | 31980637 | Decreased |
| AW492721 | 0.10209 | 0.40649 | −0.3044 | AW492721 | 7063002 | Increased |
| NM_173760 | 0.48818 | 0.3762 | 0.11198 | AW555814 | 166706912 | Decreased |
| AK053394 | 0 | −0.13334 | 0.13334 | B830012L14Rik | 26095750 | Decreased |
| AK021165 | 0.13122 | 0.2649 | −0.13368 | BC042720 | 12861959 | Increased |
| NM_013867 | −0.42999 | −0.57163 | 0.14164 | Bcar3 | 7304924 | Decreased |
| NM_009741 | 0.10226 | 0.29189 | −0.18963 | Bcl2 | 133892546 | Increased |
| NM_007554 | −0.52894 | −0.38067 | −0.14827 | Bmp4 | 121949822 | Increased |
| NM_027430 | 1.15635 | 1.06031 | 0.09604 | Brp44 | 141803058 | Decreased |
| NM_029912 | 0.30738 | 0.13701 | 0.17037 | C030022K24Rik | 22296590 | Decreased |
| AK038902 | 0.02576 | 0.29535 | −0.26959 | C130026L21Rik | 26086825 | Increased |
| AK008884 | −0.14897 | −0.26056 | 0.11159 | C1galt1 | 12843346 | Decreased |
| NM_172746 | −0.52851 | −0.46057 | −0.06794 | C86302 | 190610051 | Increased |
| NM_007581 | −0.84918 | −0.75679 | −0.09239 | Cacnb3 | 113680270 | Increased |
| AK053290 | 0.11395 | 0.34811 | −0.23416 | Camk2d | 26095687 | Increased |
| NM_178597 | 0.16363 | −0.1139 | 0.27753 | Camk2g | 85362743 | Decreased |
| NM_009829 | −0.18803 | −0.022 | −0.16603 | Ccnd2 | 80751174 | Increased |
| NM_023117 | −0.3949 | −0.50948 | 0.11458 | Cdc25b | 162329553 | Decreased |
| NM_174988 | 0.28273 | 0.02848 | 0.25425 | Cdh22 | 164698423 | Decreased |
| NM_173370 | 0.39903 | 0.24779 | 0.15124 | Cds1 | 34328392 | Decreased |
| NM_009886 | −0.15329 | −0.3211 | 0.16781 | Celsr1 | 115648152 | Decreased |
| M29010 | 0.58027 | 0.80432 | −0.22405 | Cfh | 192561 | Increased |
| M12660 | 0.47193 | 0.67525 | −0.20332 | Cfh | 193724 | Increased |
| NM_025366 | 0.35496 | 0.28609 | 0.06887 | Chchd1 | 142365360 | Decreased |
| NM_181391 | 0.54832 | 0.46749 | 0.08083 | Chchd7 | 142370747 | Decreased |
| NM_027294 | 1.17089 | 1.02455 | 0.14634 | Cklfsf8 | 142373686 | Decreased |
| AK173076 | 0.37271 | 0.19906 | 0.17365 | Cobll1 | 50510736 | Decreased |
| NM_025379 | 1.01362 | 0.93215 | 0.08147 | Cox7b | 70909318 | Decreased |
| AK041861 | 0.28765 | 0.39915 | −0.1115 | Cul5 | 26088694 | Increased |
| AK084199 | 0.10026 | 0.26685 | −0.16659 | D230007K08Rik | 26350998 | Increased |
| AK077061 | 0.74468 | 1.22004 | −0.47536 | D830007B15Rik | 26345867 | Increased |
| AK080280 | −0.1686 | 0.00244 | −0.17104 | D930036F22Rik | 26099129 | Increased |
| AK047998 | 0.39992 | 0.57951 | −0.17959 | D9Ertd809e | 26092578 | Increased |
| NM_010019 | 0.21226 | 0.37208 | −0.15982 | Dapk2 | 164565399 | Increased |
| NM_007830 | 0.24467 | 0.1552 | 0.08947 | Dbi | 83921597 | Decreased |
| NM_010023 | 1.01196 | 0.77511 | 0.23685 | Dci | 215490121 | Decreased |
| NM_007842 | −0.15609 | −0.0357 | −0.12039 | Dhx9 | 150456418 | Increased |
| NM_153550 | 0.23803 | 0.38864 | −0.15061 | Dirc2 | 118130106 | Increased |
| AK036480 | 0.09246 | 0.18344 | −0.09098 | Disp2 | 26331431 | Increased |
| NM_007876 | 0.48348 | 0.67912 | −0.19564 | Dpep1 | 161016832 | Increased |
| NM_177015 | 1.01396 | 1.28117 | −0.26721 | E130010M05Rik | 31342600 | Increased |
| NM_134111 | −0.30628 | −0.01067 | −0.29561 | Eaf2 | 40254104 | Increased |
| NM_207654 | −0.1811 | −0.07903 | −0.10207 | Efna5 | 46560569 | Increased |
| NM_010113 | 0.44016 | 0.23173 | 0.20843 | Egf | 170172523 | Decreased |
| NM_182840 | 0.06147 | 0.2441 | −0.18263 | Emilin3 | 33469054 | Increased |
| NM_010135 | 0.71752 | 0.88653 | −0.16901 | Enah | 133778925 | Increased |
| NM_010165 | −0.58168 | −0.39298 | −0.1887 | Eya2 | 118129931 | Increased |
| AK085100 | −0.01431 | −0.122 | 0.10769 | Fbxl5 | 26351458 | Decreased |
| NM_133765 | 0.31378 | 0.2265 | 0.08728 | Fbxo31 | 158517903 | Decreased |
| NM_021564 | −0.41188 | −0.25964 | −0.15224 | Fetub | 144226246 | Increased |
| AK020105 | 0.15125 | 0.30088 | −0.14963 | Flt3l | 12860586 | Increased |
| NM_020510 | −0.45378 | −0.33187 | −0.12191 | Fzd2 | 125628663 | Increased |
| K01347 | 0.27109 | 0.21843 | 0.05266 | Gfap | 193465 | Decreased |
| AF206030 | 0.00343 | 0.10444 | −0.10101 | Gm1499 | 7769334 | Increased |
| NM_010304 | 0.15899 | 0.25529 | −0.0963 | Gna15 | 34328487 | Increased |
| NM_010308 | −0.09316 | 0.07304 | −0.1662 | Gnao1 | 116325994 | Increased |
| NM_022422 | 0.00118 | 0.1831 | −0.18192 | Gng13 | 157951662 | Increased |
| BC026382 | 0.51802 | 0.37132 | 0.1467 | Gpr155 | 20071347 | Decreased |
| AK039156 | 0.46148 | 0.57488 | −0.1134 | Gprasp1 | 26333082 | Increased |
| NM_018869 | 0.42468 | 0.55688 | −0.1322 | Gprk5 | 159032058 | Increased |
| NM_182805 | 0.29678 | 0.14658 | 0.1502 | Gpt1 | 169658389 | Decreased |
| NM_173866 | −0.09024 | −0.21984 | 0.1296 | Gpt2 | 146198526 | Decreased |
| NM_010350 | 0.3485 | 0.17813 | 0.17037 | Grin2c | 75709201 | Decreased |
| NM_130455 | 0.06341 | −0.10687 | 0.17028 | Grin3b | 53759069 | Decreased |
| AY302216 | −0.04318 | 0.04714 | −0.09032 | H2-M2 | 34732747 | Increased |
| AK035316 | 0.4257 | 0.62754 | −0.20184 | Hadha | 26084523 | Increased |
| AK077185 | 0.11176 | 0.02942 | 0.08234 | Hectd1 | 26346031 | Decreased |
| AK041629 | −0.52713 | −0.33309 | −0.19404 | Hectd2 | 26334622 | Increased |
| NM_173789 | 0.3639 | 0.15809 | 0.20581 | Helt | 46048389 | Decreased |
| NM_178218 | −0.34444 | −0.02616 | −0.31828 | Hist3h2a | 142376228 | Increased |
| NM_175652 | −0.25066 | −0.196 | −0.05466 | Hist4h4 | 28316745 | Increased |
| NM_008258 | −0.43165 | −0.35835 | −0.0733 | Hn1 | 6680236 | Increased |
| NM_010470 | −0.13372 | 0.0747 | −0.20842 | Hp1bp3 | 171543868 | Increased |
| AK005714 | 0.11649 | 0.31632 | −0.19983 | Hspb9 | 12838432 | Increased |
| NM_010480 | −0.32979 | −0.11601 | −0.21378 | Hspca | 146134469 | Increased |
| AK077477 | 0.10709 | 0.29429 | −0.1872 | Igfbp3 | 26346335 | Decreased |
| NM_008344 | −0.33812 | −0.18404 | −0.15408 | Igfbp6 | 168693653 | Increased |
| NM_009349 | 0.07468 | 0.48455 | −0.40987 | Inmt | 145966708 | Increased |
| BC053524 | 0.3767 | 0.02741 | 0.34929 | Ipo7 | 31657147 | Decreased |
| NM_008389 | −0.02853 | −0.11549 | 0.08696 | Ipp | 142368919 | Decreased |
| NM_018826 | −0.0227 | 0.1087 | −0.1314 | Irx5 | 42476078 | Increased |
| NM_146145 | −0.04416 | −0.13117 | 0.08701 | Jak1 | 111607495 | Decreased |
| AK047208 | 0.03293 | −0.05786 | 0.09079 | Kif21a | 26338639 | Decreased |
| NM_010115 | 0.26785 | 0.09486 | 0.17299 | Klk13 | 85719297 | Decreased |
| NM_010644 | 0.38724 | 0.14428 | 0.24296 | K1k26 | 6754459 | Decreased |
| NM_008484 | −0.21289 | −0.32321 | 0.11032 | Lamb3 | 113865980 | Decreased |
| AK083099 | 0.34528 | 0.47518 | −0.1299 | Lip1 | 26101063 | Increased |
| AK033684 | 0.11488 | 0.21982 | −0.10494 | Lrig2 | 26083511 | Increased |
| BC025128 | −0.09832 | 0.04742 | −0.14574 | Lrrc51 | 19263880 | Increased |
| NM_177725 | 0.61596 | 0.54461 | 0.07135 | Lrrc8 | 62388880 | Decreased |
| NM_008564 | −1.13134 | −0.91037 | −0.22097 | Mcm2 | 172088118 | Increased |
| NM_201362 | 0.46172 | 0.71563 | −0.25391 | MGC54896 | 141802310 | Increased |
| NM_019914 | −0.00868 | 0.15829 | −0.16697 | Mllt11 | 141803083 | Increased |
| BC062639 | −0.02815 | 0.03409 | −0.06224 | Mon1b | 38571587 | Increased |
| NM_025301 | 0.04254 | −0.00576 | 0.0483 | Mrpl17 | 84875533 | Decreased |
| NM_011885 | 0.48303 | 0.30353 | 0.1795 | Mrps12 | 156151357 | Decreased |
| NM_175374 | 0.09737 | 0.00471 | 0.09266 | Mtrf1l | 89363036 | Decreased |
| AK008788 | 0.72565 | 0.62631 | 0.09934 | Ndufab1 | 12843196 | Decreased |
| NM_026612 | 1.0524 | 0.97223 | 0.08017 | Ndufb2 | 146141166 | Decreased |
| NM_012050 | −0.24175 | −0.0619 | −0.17985 | Omd | 218749871 | Increased |
| NM_016768 | −0.05817 | −0.12733 | 0.06916 | Pbx3 | 7949104 | Decreased |
| NM_011068 | 0.58781 | 0.45417 | 0.13364 | Pex11a | 6755033 | Decreased |
| X98848 | 0.37718 | 0.10277 | 0.27441 | Pfkfb1 | 1495705 | Decreased |
| NM_023734 | 0.85584 | 1.17745 | −0.32161 | Pi16 | 116089319 | Increased |
| BC053071 | −0.13136 | −0.3197 | 0.18834 | Polr3a | 31418570 | Decreased |
| NM_011141 | 0.24233 | 0.52445 | −0.28212 | Pou3f1 | 145279231 | Increased |
| AK122434 | 0.48024 | 0.70749 | −0.22725 | Ppm1e | 28972599 | Increased |
| NM_153745 | 0.10139 | 0.24631 | −0.14492 | Prkag3 | 118130072 | Increased |
| NM_008855 | −0.10301 | −0.22713 | 0.12412 | Prkcb1 | 116734871 | Decreased |
| NM_011171 | −0.74744 | −0.48561 | −0.26183 | Procr | 6755175 | Increased |
| NM_026662 | −0.36733 | −0.50762 | 0.14029 | Prps2 | 146141229 | Decreased |
| NM_011235 | −0.12261 | −0.01558 | −0.10703 | Rad51l3 | 127139188 | Increased |
| AK018120 | −0.03774 | 0.17471 | −0.21245 | Rasgef1a | 12857691 | Increased |
| NM_009101 | −0.07005 | −0.01095 | −0.0591 | Rras | 133891823 | Increased |
| AK015925 | 0.2214 | 0.09939 | 0.12201 | Saps3 | 12854455 | Decreased |
| NM_009201 | −0.4311 | −0.26085 | −0.17025 | Slc1a5 | 114326473 | Increased |
| NM_134420 | 0.173 | 0.07394 | 0.09906 | Slc26a6 | 158341685 | Decreased |
| NM_016917 | 0.42922 | 0.19662 | 0.2326 | Slc40a1 | 124248584 | Decreased |
| AK122369 | 1.70656 | 1.85388 | −0.14732 | Sorbs2 | 28972394 | Increased |
| AF031816 | 0.25395 | 0.48805 | −0.2341 | Sorl1 | 2654024 | Increased |
| NM_013677 | 0.12425 | 0.05192 | 0.07233 | Surf1 | 160707898 | Decreased |
| NM_178650 | 0.07493 | −0.08657 | 0.1615 | Tbc1d10c | 126517464 | Decreased |
| BC036146 | 0.75832 | 0.55592 | 0.2024 | Tfpi | 23271604 | Decreased |
| NM_028876 | −0.18137 | −0.31249 | 0.13112 | Tmed5 | 141802491 | Decreased |
| NM_019432 | 0.19279 | 0.00026 | 0.19253 | Tmem37 | 31980888 | Decreased |
| NM_145403 | 0.23539 | 0.10703 | 0.12836 | Tmprss4 | 118130127 | Decreased |
| NM_021327 | 0.00277 | 0.09427 | −0.0915 | Tnip1 | 10946635 | Increased |
| NM_020507 | 0.27546 | 0.0858 | 0.18966 | Tob2 | 108796647 | Decreased |
| NM_172570 | 0.05805 | 0.15376 | −0.09571 | Trim47 | 148747457 | Increased |
| NM_019656 | 0.11723 | −0.09515 | 0.21238 | Tspan6 | 125490374 | Decreased |
| BC038619 | 0.19032 | 0.2838 | −0.09348 | Tspan9 | 24047235 | Increased |
| NM_178869 | −0.32367 | 0.03524 | −0.35891 | Ttll1 | 188219534 | Increased |
| NM_011677 | 0.08471 | −0.03817 | 0.12288 | Ung | 101943420 | Decreased |
| NM_028866 | −0.08317 | −0.24839 | 0.16522 | Wdr33 | 21362284 | Decreased |
| NM_016873 | −0.20723 | 0.11703 | −0.32426 | Wisp2 | 8394540 | Increased |
| NM_175638 | 0.09861 | −0.05011 | 0.14872 | Wnk4 | 66793432 | Decreased |
| AK031247 | −0.13726 | −0.20132 | 0.06406 | Xrn2 | 26327148 | Decreased |

TABLE 3

| | Expression Level | | | |
|---|---|---|---|---|
| | of Mouse Gene in | Gene Name | GI Number | |
| Gene name | Mutant Compared | (human | (human | miRs which are predicted to |
| (mouse) | to Wild-Type | ortholog) | ortholog) | target this gene |
| Acaa2 | Decreased | ACAA2 | 167614484 | miR-27b |
| Acta2 | Increased | ACTA2 | 213688378 | miR-27b |
| Actr10 | Decreased | ACTR10 | 7689030 | miR-125b |
| Aldob | Decreased | ALDOB | 28419 | miR-499 |
| 2610040C18Rik | Increased | APITD1 | 41327702 | miR-29c, miR-27b |
| Bcar3 | Decreased | BCAR3 | 3237305 | miR-185 |
| C1galt1 | Decreased | C1GALT1 | 187608326 | miR-152 |
| MGC54896 | Increased | CCDC68 | 219689134 | miR-499, miR-210, miR-378, |
| | | | | miR-497, miR-27b, miR-328 |
| Ccnd2 | Increased | CCND2 | 209969683 | miR-497 |
| Cdh22 | Decreased | CDH22 | 16753220 | miR-152 |
| Cfh | Increased | CFH | 184172390 | miR-185, miR-328 |
| E130010M05Rik | Increased | COL4A4 | 116256355 | miR-29c |
| 2810437L13Rik | Increased | COX19 | 110349770 | miR-27b, miR-378, miR-125b, |
| | | | | miR-328, miR-24, miR-185, |
| | | | | miR-199a, miR-199b |
| Dapk2 | Increased | DAPK2 | 71774012 | miR-208, miR-499 |
| Dci | Decreased | DCI | 62530383 | miR-208, miR-499 |
| Disp2 | Increased | DISP2 | 25121979 | miR-185 |
| Eaf2 | Increased | EAF2 | 41350199 | miR-208, miR_499 |
| Efna5 | Increased | EFNA5 | 1019430 | miR-199a, miR-199b |
| Egf | Decreased | EGF | 31120 | miR-499, miR-199a, mir-199b |
| Enah | Increased | ENAH | 56549692 | miR-497 |
| Fbxo31 | Decreased | FBXO31 | 217272875 | miR-210, miR-27b, miR-29c, |
| | | | | miR-378 |
| Fetub | Increased | FETUB | 58331239 | miR-24 |
| Gfap | Decreased | GFAP | 196115280 | miR-125b, miR-130a, miR-185, |
| | | | | miR-199a, miR199b, miR-24, |
| | | | | miR-497 |
| Gna15 | Increased | GNA15 | 156104882 | miR-210, miR-185 |
| Gng13 | Increased | GNG13 | 157951661 | miR-27b |
| Gpr155 | Decreased | GPR155 | 74315998 | miR-24, miR-30c, miR-30d, miR |
| | | | | 378, miR-27b |
| Grin2c | Decreased | GRIN2C | 55770853 | miR-328 |
| Hectd1 | Decreased | HECTD1 | 71891694 | miR-497 |
| Igfbp6 | Increased | IGFBP6 | 49574524 | miR-199a, miR-199b |
| Inmt | Increased | INMT | 66933017 | miR-24, miR-199a, miR-199b, |
| | | | | miR-152, miR-328, miR-486 |
| Lamb3 | Decreased | LAMB3 | 62868214 | miR-24 |
| Lrrc51 | Increased | LRTOMT | 223718146 | miR- 24, miR-499, miR-199a, |
| | | | | miR-199b |
| Mcm2 | Increased | MCM2 | 468703 | miR-378 |
| A930031D07Rik | Decreased | MFSD4 | 170932531 | miR-199a, miR-199b, miR-210, |
| | | | | miR-24 |
| Mllt11 | Increased | MLLT11 | 55774979 | miR-29c |
| Mon1b | Increased | MON1B | 38016939 | miR-125b, miR-497, miR-27b, |
| | | | | miR-185 |
| Mtrf1l | Decreased | MTRF1L | 166795302 | miR-378 |
| D230007K08Rik | Increased | NLRC3 | 118918428 | miR- 99a, miR-328, miR-486, |
| | | | | miR-24, miR-199a, miR-199b, |
| | | | | miR-185 |
| Omd | Increased | OMD | 176865970 | miR-378, miR-199a, miR-199b |
| Polr3a | Decreased | POLR3A | 39725937 | miR-24, miR-199a, miR-199b |
| Ppm1e | Increased | PPM1E | 5689480 | miR-29c |
| Prkag3 | Increased | PRKAG3 | 8215681 | miR-328, miR-185, miR-24 |
| Procr | Increased | PROCR | 565267 | miR-185, miR-27b |
| Rad51l3 | Increased | RAD51L3 | 217416414 | miR-30c, miR-30d, miR-497 |
| Saps3 | Decreased | SAPS3 | 55925644 | miR-497 |
| Slc26a6 | Decreased | SLC26A6 | 20336275 | miR-29c, miR-125b, miR-378 |
| Tbc1d10c | Decreased | TBC1D10C | 48762676 | miR-125b |
| Tfpi | Decreased | TFPI | 98991770 | miR-199a, miR-199b, miR-24, |
| | | | | miR-27b |
| C030022K24Rik | Decreased | TMEM116 | 24308397 | miR-185, miR-125b |
| Tmem37 | Decreased | TMEM37 | 116325982 | miR-125b, miR-328 |
| Tspan6 | Decreased | TSPAN6 | 2832292 | miR-199a, miR-199b |
| Ung | Decreased | UNG | 19718750 | miR-497, miR-199a, miR-199b |
| Wdr33 | Decreased | WDR33 | 56243589 | miR-199a, miR-199b, miR- |
| | | | | 130a, miR-152, miR-208, miR- |
| | | | | 27b, miR-29c, miR-30c, miR- |
| | | | | 30d, miR-497, miR-499 |
| Wisp2 | Increased | WISP2 | 18491001 | miR-185 |

What is claimed is:

1. A method of diagnosing hypertrophic cardiomyopathy comprising, a) measuring the level of expression of a miRNA from Table 1, or an ortholog thereof, in a heart sample from a subject, and b) comparing the level of expression of said miRNA with that of normal heart tissue, wherein if the level of expression of said miRNA in the subject sample is different to the level of expression of said miRNA in normal heart tissue, the subject is determined to have hypertrophic cardiomyopathy.

2. The method of claim 1 wherein the level of expression of said miRNA in the subject sample is lower than the level of expression of said miRNA in normal heart tissue and wherein said miRNA is selected from the group consisting of the human ortholog of mmu-miR-709, the human ortholog of mmu-miR-290, hsa-miR-208a, hsa-miR-185, hsa-miR-30d, hsa-miR-30c, hsa-miR-499, and hsa-miR-29c.

3. The method of claim 1 wherein the level of expression of said miRNA in the subject sample is higher than the level of expression of said miRNA in normal heart tissue and wherein said miRNA is selected from the group consisting of hsa-miR-378, hsa-miR-99a, hsa-miR-125b, hsa-miR-199a-3p, hsa-miR-199b, hsa-miR-486, hsa-miR-497, hsa-miR-328, hsa-miR-210, hsa-miR-24, hsa-miR-130a, hsa-miR-27b, hsa-miR-199a-5p, and hsa-miR-152.

4. A method of diagnosing hypertrophic cardiomyopathy comprising, a) measuring the level of expression of a gene from Tables 2-3, or an ortholog thereof, in a heart sample from a subject and b) comparing the level of expression of said gene with that of normal heart tissue, wherein if the level of expression of said gene in the subject sample is different to the level of expression of said gene in normal heart tissue, the subject is determined to have hypertrophic cardiomyopathy.

5. The method of claim 4 wherein the level of expression of said gene in the subject sample is lower than the level of expression of said gene in normal heart tissue and wherein said gene is selected from the group consisting of ACAA2, ACTR10, ALDOB, BCAR3, C1GALT1, CDH22, DCI, EGF, FBXO31, GFAP, GPR155, GRIN2C, HECTD1, LAMB3, MFSD4, MTRF1L, POLR3A, SAPS3, SLC26A6, TBC1D10C, TFPI, TMEM116, TMEM37, TSPAN6, UNG, and WDR33.

6. The method of claim 4 wherein the level of expression of said gene in the subject sample is higher than the level of expression of said gene in normal heart tissue and wherein said gene is selected from the group consisting of ACTA2, APITD1, CCDC68, CCND2, CFH, COL4A4, COX19, DAPK2, DISP2, EAF2, EFNA5, ENAH, FETUB, GNA15, GNG13, IGFBP6, INMT, LRTOMT, MCM2, MLLT11, MON1B, NLRC3, OMD, PPM1E, PRKAG3, PROCR, RAD51L3, and WISP2.

7. A method of treating hypertrophic cardiomyopathy comprising a) identifying a subject suspected of having hypertrophic cardiomyopathy, and b) inhibiting the expression or activity of a miRNA selected from the group consisting of hsa-miR-378, hsa-miR-99a, hsa-miR-125b, hsa-miR-199a-3p, hsa-miR-199b, hsa-miR-486, hsa-miR-497, hsa-miR-328, hsa-miR-210, hsa-miR-24, hsa-miR-130a, hsa-miR-27b, hsa-miR-199a-5p, and hsa-miR-152 in the heart cells of said subject.

8. The method of claim 7 wherein a miRNA inhibitor is used to inhibit the activity of said miRNA.

9. A method of treating hypertrophic cardiomyopathy comprising a) identifying a subject suspected of having hypertrophic cardiomyopathy, and b) increasing the level of a miRNA selected from the group consisting of the human ortholog of mmu-miR-709, the human ortholog of mmu-miR-290, hsa-miR-208a, hsa-miR-185, hsa-miR-30d, hsa-miR-30c, hsa-miR-499, and hsa-miR-29c in the heart cells of said subject.

10. The method of claim 9 wherein a miRNA mimic is used to increase the level of said miRNA.

11. A method of treating hypertrophic cardiomyopathy comprising a) identifying a subject suspected of having hypertrophic cardiomyopathy, and b) inhibiting the expression or activity of a gene selected from the group consisting of ACTA2, APITD1, CCDC68, CCND2, CFH, COL4A4, COX19, DAPK2, DISP2, EAF2, EFNA5, ENAH, FETUB, GNA15, GNG13, IGFBP6, INMT, LRTOMT, MCM2, MLLT11, MON1B, NLRC3, OMD, PPM1E, PRKAG3, PROCR, RAD51L3, and WISP2 in heart cells of said subject.

12. A method of treating hypertrophic cardiomyopathy comprising a) identifying a subject suspected of having hypertrophic cardiomyopathy, and b) increasing the expression or activity of a gene selected from the group consisting of ACAA2, ACTR10, ALDOB, BCAR3, C1GALT1, CDH22, DCI, EGF, FBXO31, GFAP, GPR155, GRIN2C, HECTD1, LAMB3, MFSD4, MTRF1L, POLR3A, SAPS3, SLC26A6, TBC1D10C, TFPI, TMEM116, TMEM37, TSPAN6, UNG, and WDR33 in heart cells of said subject.
